(19) United States
(12) Patent Application Publication
(10) Pub. No.: US 2008/0103504 A1
(43) Pub. Date: May 1, 2008

(54) PERCUTANEOUS SPINAL STENOSIS TREATMENT

(76) Inventors: Gregory P. SCHMITZ, Los Gatos, CA (US); Jeffery L. Bleich, Palo Alto, CA (US); Eric C. Miller, Los Gatos, CA (US); Scott M. Smith, Redwood Shores, CA (US)

Correspondence Address: SHAY GLENN LLP, 2755 CAMPUS DRIVE, SUITE 210, SAN MATEO, CA, 94403, US

(21) Appl. No.: 11/870,370

(22) Filed: Oct. 10, 2007

Related U.S. Application Data
(60) Provisional application No. 60/863,544, filed on Oct. 30, 2006.

(51) Int. Cl. A61B 17/32; A61B 18/14; A61N 1/36

(52) U.S. Cl. 606/79; 606/45; 607/117

(57) ABSTRACT

A method for percutaneously removing ligamentum flavum tissue in a spine to treat spinal stenosis may involve percutaneously advancing a distal portion of a tissue removal cannula into the ligamentum flavum tissue, uncovering a side-opening aperture disposed on the distal portion of the cannula to expose a tissue cutter disposed in the cannula, and cutting ligamentum flavum tissue using the tissue cutter while the aperture is uncovered. A device for percutaneously removing ligamentum flavum tissue in a spine to treat spinal stenosis may include a cannula including a side-facing aperture, an aperture cover slidably coupled with the cannula and configured to advance and retract to cover and uncover the aperture, and a tissue cutter slidably disposed within the cannula and configured to extend through the aperture to cut ligamentum flavum tissue.

### CROSS REFERENCE TO RELATED APPLICATIONS

This application claims priority to U.S. Provisional Patent Application Ser. No. 60/863,544 (Attorney Docket No. 10376-710.101), entitled “Percutaneous Spinal Stenosis Treatment,” and filed Oct. 30, 2006, the full disclosure of which is hereby incorporated by reference.

### FIELD OF THE INVENTION

The present invention relates generally to medical/surgical devices and methods. More specifically, the present invention relates to devices and methods for spinal stenosis treatment.

### BACKGROUND OF THE INVENTION

In recent years, less invasive (or “minimally invasive”) surgical techniques have become increasingly more popular, as physicians, patients and medical device innovators have sought to reduce the trauma, recovery time and side effects typically associated with conventional surgery. Developing less invasive surgical methods and devices, however, poses many challenges. For example, less invasive techniques typically involve working in a smaller operating field, working with smaller devices, and trying to operate with reduced or even no direct visualization of the structures being treated. These challenges are often compounded when target tissues of a given procedure reside very close to one or more vital, non-target tissues.

One area of surgery which would likely benefit from the development of less invasive techniques is the treatment of spinal stenosis. Spinal stenosis occurs when nerve tissue and/or the blood vessels supplying nerve tissue in the spine become impinged by one or more structures the lower (or lumbar) spine and can cause severe pain, numbness and/or loss of function in the lower back and/or one or both lower limb.

FIG. 1 is a top view of a vertebra with the cauda equina (the bundle of nerves that extends from the base of the spinal cord) shown in cross section and two nerve roots branching from the cauda equina to exit the central spinal canal and extend through intervertebral foramina on either side of the vertebra. Spinal stenosis can occur when the spinal cord, cauda equina and/or nerve root(s) are impinged by one or more tissues in the spine, such as buckled or thickened ligamentum flavum, hypertrophied facet joint (shown as superior articular processes shown in FIG. 1), osteophytes (or “bone spurs”) on vertebrae, spondylolisthesis (sliding of one vertebra relative to an adjacent vertebra), facet joint synovial cysts, and/or collapse, bulging or herniation of an intervertebral disc. Impingement of neural and/or neurovascular tissue in the spine by one or more of these tissues may cause pain, numbness and/or loss of strength or mobility in one or both of a patient's lower limbs and/or of the patient's back.

In the United States, spinal stenosis occurs with an incidence of between 4% and 6% of adults aged 50 and older and is the most frequent reason cited for back surgery in patients aged 60 and older. Patients suffering from spinal stenosis are typically first treated with conservative approaches such as exercise therapy, analgesics, anti-inflammatory medications, and epidural steroid injections. When these conservative treatment options fail and symptoms are severe, as is frequently the case, surgery may be required to remove impinging tissue and decompress the impinged nerve tissue.

Lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Therefore, it would be desirable to provide less invasive surgical methods and devices for treating spinal stenosis. For example, it would be desirable to method and device for removing impinging tissue from a spine percutaneously, or at least with a minimally invasive incision, while maintaining safety and preventing damage to non-target tissues. At least some of these objectives will be met by the present invention.

### SUMMARY OF THE INVENTION

In one aspect of the present invention, a method for percutaneously removing ligamentum flavum tissue in a spine to treat spinal stenosis may involve: percutaneously advancing a distal portion of a tissue removal cannula into the ligamentum flavum tissue; uncovering a side-opening aperture disposed on the distal portion of the cannula to expose a tissue cutter disposed in the cannula; and cutting ligamentum flavum tissue using the tissue cutter while the aperture is uncovered. In some embodiments, uncovering the aperture may involve retracting an inner cannula through the tissue removal cannula. Cutting ligamentum flavum tissue may involve cutting tissue using a tissue cutter selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices.

In some embodiments, the ligamentum flavum tissue may be cut using a radiofrequency device, and the method further involves, before the uncovering step, activating the radiofrequency device. In some embodiments, the method may include, before the uncovering step: articulating the distal portion of the cannula relative to the proximal portion; and advancing the articulated distal portion at least partway into an intervertebral foramen of the spine. In some embodiment, the method may further involve extending the cutter out of the aperture before the cutting step.

Optionally, the method may include removing the cut ligamentum flavum tissue through the cannula. In some embodiments, removing the cut tissue comprises applying suction to the cannula. In some embodiments, removing the cut tissue includes: engaging the cut tissue with the tissue cutter or a separate tissue engaging member; and retracting the tissue cutter or tissue engaging member through the cannula. Some embodiments may further involve introducing a substance through the side-facing aperture of the cannula, the substance selected from the group consisting of a hemostatic agent, an analgesic, an anesthetic and a steroid.

Optionally, some embodiments of the method may include, before the cutting step: activating a nerve stimulator coupled with the distal portion of the cannula; and monitoring for response to the activation. Some embodiments of the method may also include deploying a shield between the cannula and non-target tissue before the cutting step. In one embodiment, the method may also include, before the cutting step: activating a nerve stimulator coupled with the shield; and monitoring for response to the activation.

In another aspect of the present invention, a method for percutaneously removing ligamentum flavum tissue in a spine to treat spinal stenosis may involve: percutaneously advancing a distal portion of a tissue removal cannula into the ligamentum flavum tissue; activating at least a first nerve stimulator coupled with the distal portion of the cannula; monitoring for response to the activation; uncovering a side-opening aperture disposed on the distal portion of the cannula to expose a tissue engaging member disposed in the cannula; engaging ligamentum flavum tissue with the tissue engaging member; and cutting ligamentum flavum tissue with a tissue cutter disposed in or on the cannula.

In some embodiments, the method may include, before the uncovering step: activating at least a second nerve stimulator coupled with the distal portion of the cannula apart from the first nerve stimulator; monitoring for response to activation; and comparing an amount of activation required to illicit a response using the first nerve stimulator with an amount of activation required to illicit a response using the second nerve stimulator. In some embodiments, cutting the ligamentum flavum tissue may involve advancing an inner cannula having a sharp distal end and disposed around the tissue engaging member and within the tissue removal cannula.

In another aspect of the present invention, a method for percutaneously removing ligamentum flavum tissue in a spine to treat spinal stenosis may involve: coupling a flexible distal portion of a tissue removal cannula with one end of a guidewire; pulling the flexible distal portion into the ligamentum flavum tissue by pulling the guidewire; uncovering a side-opening aperture disposed on the distal portion of the cannula to expose a tissue cutter disposed in the cannula; and cutting ligamentum flavum tissue using the tissue cutter.

In some embodiments, the method may further include applying tensioning force to the tissue removal cannula and the guidewire, before the cutting step, to urge the aperture against the ligamentum flavum tissue. The method may optionally further involve, before the cutting step: activating a nerve stimulator coupled with the distal portion of the cannula; and monitoring for response to the activation. In some embodiments, the method may also include deploying a shield between the cannula and non-target tissue before the cutting step. Optionally, the method may include, before the cutting step: activating a nerve stimulator coupled with the shield; and monitoring for response to the activation.

In another aspect of the present invention, a method for percutaneously removing ligamentum flavum tissue in a spine to treat spinal stenosis may involve: percutaneously advancing a distal portion of a tissue removal device into at least one of an epidural space or a ligamentum flavum of the spine; activating an energy delivery member disposed on or in the distal portion of the tissue removal device; and cutting ligamentum flavum tissue with the activated energy delivery member.

In some embodiments, advancing the distal portion may involve pulling the distal portion behind a guidewire. In some embodiments, the distal portion may be advanced at least partway into an intervertebral foramen of the spine. In some embodiments, the distal portion of the tissue removal device may be flexible. In some embodiments, a proximal portion extending proximally from the distal portion of the tissue removal device may be flexible. In some embodiments, activating the energy delivery member may involve activating a member selected from the group consisting of electrosurgical devices, bipolar electrodes, monopolar electrodes, thermal electrodes, cold ablation devices, lasers, ultrasound devices and cryogenic devices. In some embodiments, cutting the tissue involves retracting the energy delivery member through tissue. In some embodiments, cutting the tissue may involve advancing the energy delivery member through tissue. Some embodiments may further involve extending the energy delivery member out of the tissue removal device before the cutting step. Some embodiments may further involve removing the cut ligamentum flavum tissue through a lumen in the tissue removal device. In some embodiments, removing the cut tissue may involve applying suction to the tissue removal device. In some embodiments, removing the cut tissue may involve: engaging the cut tissue with the energy delivery member or a separate tissue engaging member; and retracting the energy delivery member or tissue engaging member through the tissue removal device.

Some embodiments may further involve introducing a substance through an aperture in the tissue removal device, the substance selected from the group consisting of a hemostatic agent, an analgesic, an anesthetic and a steroid. Some embodiments may involve, before the cutting step: activating at least a first nerve stimulator coupled with the distal portion of the tissue removal device; and monitoring for response to the activation. Some embodiments may involve, before the cutting step: activating at least a second nerve stimulator coupled with the distal portion of the tissue removal device apart from the first nerve stimulator; monitoring for response to activation; and comparing an amount of activation required to illicit a response using the first nerve stimulator with an amount of activation required to illicit a response using the second nerve stimulator. Optionally, the method may also involve automatically deactivating the energy delivery member if the response to activation by the nerve stimulator(s) indicates that the energy delivery member is in contact with or near nerve tissue. The method may also include repeating the activating and monitoring steps during the cutting step; and repeating the automatic deactivating step whenever the response to activation indicates that the energy delivery member is in contact with or near nerve tissue. In one embodiment, the method may include deploying a shield between the cannula and non-target tissue before the cutting step. Such a method may also include, before the cutting step: activating at least a first nerve stimulator coupled with the shield; and monitoring for response to the activation. Such a method may also include, before the cutting step: activating at least a second nerve stimulator coupled with the shield apart from the first nerve stimulator; monitoring for response to activation; and comparing an amount of activation required to illicit a response using the first nerve stimulator with an amount of activation required to illicit a response using the second nerve stimulator. In some embodiments, the method also may include automatically deactivating the energy delivery member if the response to activation by the nerve stimulator(s) indicates that the energy delivery member is in contact with or near nerve tissue. In one embodiment, the method may also include: repeating the activating and monitoring steps during the cutting step; and repeating the automatic deactivating step whenever the response to activation indicates that the energy delivery member is in contact with or near nerve tissue.

In another aspect of the present invention, a device for percutaneously removing ligamentum flavum tissue in a spine to treat spinal stenosis may include: a cannula having a proximal end, a tissue-penetrating distal end, and a side-facing aperture closer to the distal end than the proximal end; an aperture cover slidably coupled with the cannula and configured to advance and retract to cover and uncover the aperture; and a tissue cutter slidably disposed within the cannula and configured to extend through the aperture to cut ligamentum flavum tissue. In some embodiments, the aperture cover may comprise an inner cannula slidably disposed in the tissue removal cannula. In some embodiments, a distal portion of the cannula may be articulatable relative to a proximal portion of the cannula.

In various embodiments, the tissue cutter may be selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices. In some embodiments, the tissue cutter may be configured to extend out of the aperture. In some embodiments, the tissue cutter may be configured to engage cut ligamentum flavum tissue and to be retracted through the cannula to remove the engaged tissue.

Optionally, the device may also include a suction connector for coupling the proximal end of the cannula with a suction device for removing cut tissue through the cannula. Also optionally, the device may include at least a first nerve stimulator coupled with the cannula at or near the aperture. Such a device may also include at least a second nerve stimulator coupled with the cannula, where the first nerve stimulator is disposed generally on the same side of the cannula as the aperture and the second nerve stimulator is disposed between about 90 degrees and about 180 degrees away from the first stimulator along a circumference of the cannula. Some embodiments may also include a shield coupled with the cannula for preventing the cutter from contacting non-target tissue.

In another aspect of the present invention, a device for percutaneously removing ligamentum flavum tissue in a spine to treat spinal stenosis may include: a cannula having a proximal end, a tissue-penetrating distal end, and a side-facing aperture closer to the distal end than the proximal end; a tissue-engaging member disposed within the cannula and adapted to engage tissue via the aperture; an aperture cover slidably coupled with the cannula and configured to advance and retract to cover and uncover the aperture, the cover having a sharp, tissue cutting edge to cut tissue engaged by the tissue-engaging member; and a nerve stimulation member coupled with the cannula adjacent or near the aperture. In some embodiments, a distal portion of the cannula may be articulatable relative to a proximal portion of the cannula. In various embodiments, the tissue-engaging member is selected from the group consisting of needles, hooks, blades, teeth and barbs. The tissue-engaging member may be slidably disposed within the cannula such that it can be retracted through the cannula to remove cut tissue from the cannula.

The aperture cover may comprise an inner cannula slidably disposed in the outer cannula. Optionally, the device may include a suction connector for coupling the proximal end of the cannula with a suction device for removing cut tissue through the cannula. Some embodiments may also include at least a second nerve stimulator coupled with the cannula apart from the first nerve stimulator. The device may further include a shield coupled with the cannula for preventing the cutter from contacting non-target tissue. The device may optionally further include a nerve stimulator coupled with the shield.

In another aspect of the present invention, a device for percutaneously removing ligamentum flavum tissue in a spine to treat spinal stenosis may include: an elongate body having a proximal portion, a flexible distal portion, and a side-facing aperture disposed on the distal portion, wherein the distal portion is configured to be passed percutaneously into at least one of an epidural space or a ligamentum flavum of the spine; and an energy delivery member disposed within the elongate body and configured to extend through the aperture to cut ligamentum flavum tissue. In some embodiments, the proximal portion of the body may be at least partially flexible. Alternatively, the proximal portion of the body may be rigid. In some embodiments, the distal portion of the body may be configured to be passed at least partway into an intervertebral foramen of the spine.

The device may further include a guidewire coupling member disposed on the distal portion of the elongate body for pulling the distal portion into the spine. In some embodiments, the energy delivery member may be selected from the group consisting of electrosurgical devices, bipolar electrodes, monopolar electrodes, thermal electrodes, cold ablation devices, lasers, ultrasound devices and cryogenic devices. In some embodiments, the energy delivery member may be slidably disposed within the elongate body and is configured to be advanced through the aperture. In one embodiment, the energy delivery member may comprise a wire loop electrode. In some embodiments, the elongate body may further include a lumen through which cut ligamentum flavum tissue may be removed.

Some embodiments may further include a suction device couplable with the elongate body for removing the cut ligamentum flavum tissue through the lumen. Some embodiments may further include an irrigation device couplable with the elongate body for passing fluid through the lumen. Some embodiments may further include a substance disposed in the lumen for delivery through the aperture, where the substance may be selected from the group consisting of a hemostatic agent, an analgesic, an anesthetic and a steroid.

The device may optionally include at least a first nerve stimulator coupled with the distal portion of the elongate body. In some embodiments, the device may also include at least a second nerve stimulator coupled with the distal portion of the elongate body apart from the first nerve stimulator. The device may also include means for detecting stimulation of a nerve. The device may also include means for automatically deactivating the energy delivery member if the means for detecting stimulation indicates that the energy delivery member is in contact with or near nerve tissue.

In some embodiments, the device may include a shield coupled with the elongate body for preventing the energy delivery member from contacting non-target tissue. In some embodiments, the device may include at least a first nerve stimulator coupled with the shield. The device may also include at least a second nerve stimulator coupled with the shield apart from the first nerve stimulator. Optionally, the device may include means for detecting stimulation of a nerve. The device may also include means for automatically deactivating the energy delivery member if the means for detecting indicates that the energy delivery member is in contact with or near nerve tissue.

In another aspect of the present invention, a system for percutaneously removing ligamentum flavum tissue in a spine to treat spinal stenosis may include: a tissue removal device, comprising: an elongate body having a proximal portion, a flexible distal portion, and a side-facing aperture disposed on the distal portion, wherein the distal portion is configured to be passed percutaneously into at least one of an epidural space or a ligamentum flavum of the spine; and an energy delivery member disposed within the elongate body and configured to extend through the aperture to cut ligamentum flavum tissue; and an energy source removably couplable with the tissue removal device for supplying energy to the energy delivery member. The tissue removal device may include any of the features and configurations described above.

Optionally, the system may also include a guidewire configured to couple with the guidewire coupling member. The system may further include a handle removably couplable with the guidewire for pulling the guidewire from outside a patient. In some embodiments, the energy delivery member may be selected, for example, from the group consisting of electrosurgical devices, bipolar electrodes, monopolar electrodes, thermal electrodes, cold ablation devices, lasers, ultrasound devices and cryogenic devices. In some embodiments, the energy source may be selected from the group consisting of a radiofrequency device, a heating device, a cooling device, a cryogenic device, a laser and an ultrasound generator.

The system may optionally further include a suction device for removing the cut ligamentum flavum tissue through the lumen. The system may optionally include an irrigation device for passing fluid through the lumen. The system may further include a substance disposed in the lumen of the tissue removal device for delivery through the aperture, wherein the substance is selected from the group consisting of a hemostatic agent, an analgesic, an anesthetic and a steroid.

The system may further include one or more nerve stimulation members, such as those described above. Optionally, the system may include means for detecting stimulation of a nerve. In some embodiments, the system may automatically deactivate the tissue removal device when nerve stimulation is detected. In some embodiments, nerve stimulators may be powered by the energy source, and means for detecting stimulation and the means for automatically deactivating the energy delivery member are coupled with the energy source.

These and other aspects and embodiments are described more fully below in the Detailed Description, with reference to the attached Drawings.

### BRIEF DESCRIPTION OF THE DRAWINGS

FIG. 1 is a cross-sectional view of a spine, showing a top view of a lumbar vertebra, a cross-sectional view of the cauda equina, and two exiting nerve roots;

FIGS. 2A-2D are cross-sectional views of a portion of a spine and back, demonstrating a percutaneous method for removing ligamentum flavum tissue to treat spinal stenosis and/or neural/neurovascular impingement, according to one embodiment of the present invention;

FIGS. 3A and 3B are top and cross-sectional views, respectively, of a device for removing ligamentum flavum tissue to treat spinal stenosis and/or neural/neurovascular impingement, according to one embodiment of the present invention;

FIGS. 4A-4E are cross-sectional views of a distal portion of a device for removing ligamentum flavum tissue to treat spinal stenosis and/or neural/neurovascular impingement, according to one embodiment of the present invention;

FIGS. 5A-5E are cross-sectional views of a distal portion of a device for removing ligamentum flavum tissue to treat spinal stenosis and/or neural/neurovascular impingement, according to an alternative embodiment of the present invention;

FIGS. 5F and 5G are side and cross-sectional views of the portion of the device from FIGS. 5A-5E;

FIGS. 6A-6E are cross-sectional views of a distal portion of a device for removing ligamentum flavum tissue to treat spinal stenosis and/or neural/neurovascular impingement, according to an alternative embodiment of the present invention;

FIG. 7 is a perspective view of a distal portion of a powered mechanical device for removing ligamentum flavum tissue to treat spinal stenosis and/or neural/neurovascular impingement, according to one embodiment of the present invention;

FIG. 8 is a perspective view of a distal portion of a powered mechanical device for removing ligamentum flavum tissue to treat spinal stenosis and/or neural/neurovascular impingement, according to an alternative embodiment of the present invention;

FIGS. 9A-9B are top and side views, respectively, of a distal portion of a powered mechanical device for removing ligamentum flavum tissue to treat spinal stenosis and/or neural/neurovascular impingement, according to an alternative embodiment of the present invention;

FIG. 10 is a cross-sectional view of a portion of a spine and back and a flexible tissue modification device in place for removing ligamentum flavum tissue, according to one embodiment of the present invention;

FIG. 11 is a cross-sectional view of a portion of a spine and back and an articulating tissue modification device in place for removing ligamentum flavum tissue, according to an alternative embodiment of the present invention;

FIG. 12A is a cross-sectional view of a portion of a spine and back and a flexible tissue modification device in place for removing ligamentum flavum tissue, according to an alternative embodiment of the present invention;

FIGS. 12B-12D are perspective views of portions of the device of FIG. 12A, in greater magnification;

FIG. 13 is a cross-sectional view of a portion of a spine and back and a flexible, non-powered mechanical tissue modification device in place for removing ligamentum flavum tissue, according to one embodiment of the present invention;

FIG. 14 is a cross-sectional view of a portion of a spine and back and a flexible tissue access device in place, with multiple optional tissue removal tools for removing ligamentum flavum tissue, according to an alternative embodiment of the present invention;

FIGS. 15A-15E are perspective and cross-sectional views of a tissue barrier device and delivery device, according to one embodiment of the present invention;

FIGS. 16A and 16B are perspective views of a tissue barrier device, delivery device and tissue modification device, according to an alternative embodiment of the present invention;

FIGS. 17A and 17B are perspective views of a tissue barrier device, delivery device and tissue modification device, according to an alternative embodiment of the present invention;

FIG. 18 is a perspective view of a tissue barrier device, delivery device and tissue modification device, according to an alternative embodiment of the present invention;

FIG. 19 is a perspective view of a tissue barrier device, delivery device and tissue modification device, according to an alternative embodiment of the present invention;

FIG. 20 is a cross-sectional view of a tissue barrier device, according to one embodiment of the present invention;

FIG. 21 is a cross-sectional view of a tissue barrier device, according to an alternative embodiment of the present invention;

FIG. 22 is a cross-sectional view of a spine with a ligamentum flavum retracting device in place, according to one embodiment of the present invention;

FIG. 23 is a cross-sectional view of a spine with a ligamentum flavum retracting device in place, according to an alternative embodiment of the present invention;

FIGS. 24A-24P are cross-sectional views of a portion of a spine and back, demonstrating a percutaneous method for removing ligamentum flavum tissue, according to one embodiment of the present invention;

FIGS. 25A-25C are cross-sectional and perspective views of a tissue barrier and needlette tissue removal device, according to one embodiment of the present invention;

FIG. 26A is a perspective view of a tissue barrier and needlette tissue removal device, according to an alternative embodiment of the present invention; and

FIG. 26B is a perspective view of a tissue barrier and needlette tissue removal device, according to an alternative embodiment of the present invention.

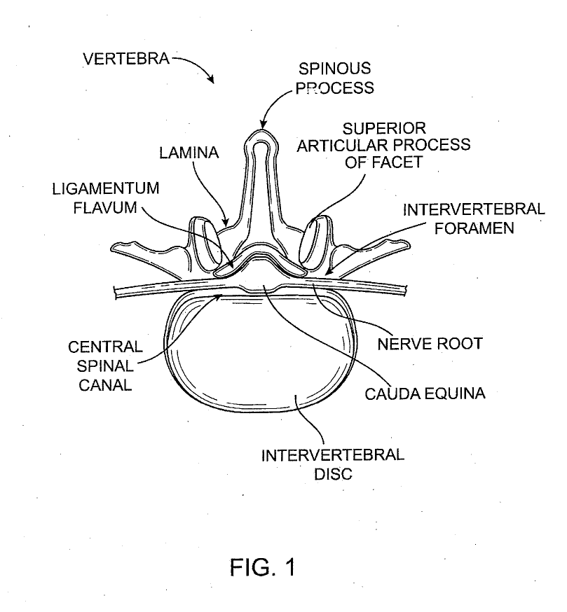

### DETAILED DESCRIPTION OF THE INVENTION

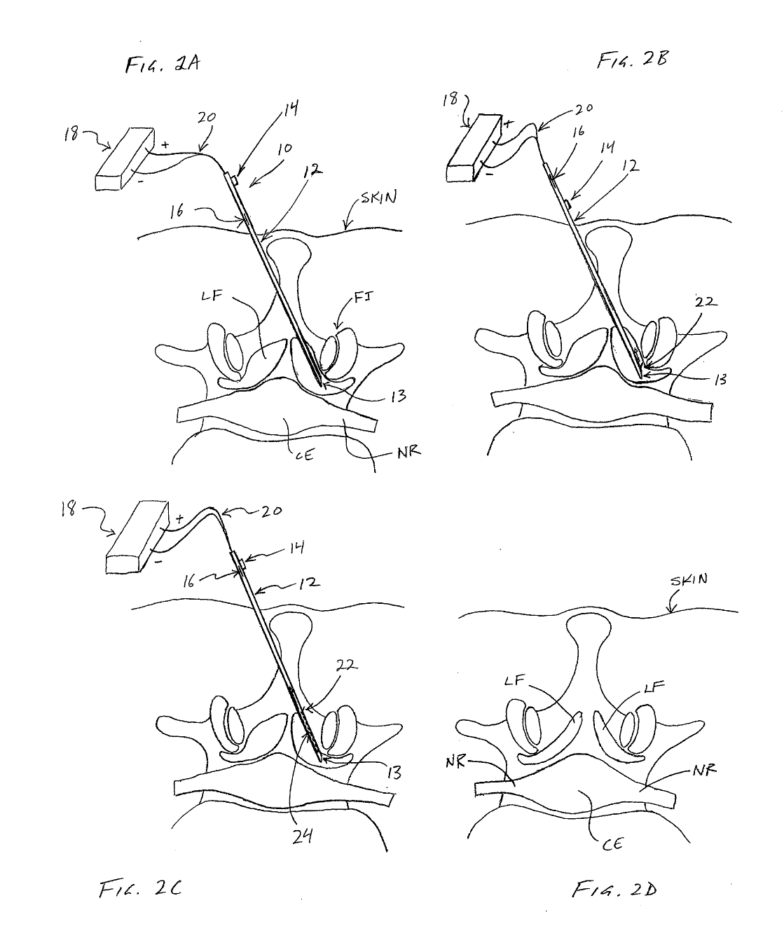

Referring to FIGS. 2A-2D, one embodiment of a method for removing ligamentum flavum (LF) tissue from a patient's spine is demonstrated. In FIGS. 2A-2D, a partial top view of a vertebra is shown, including ligamentum flavum (LF), facet joint (FJ), nerve root (NR) and cauda equina (CE). The patient's skin is also shown, although none of the anatomical structures, nor the various devices used therein, are necessarily drawn to scale.

In one embodiment, referring to FIG. 2A, a tissue removal device 10 may be advanced percutaneously through a patient's skin to position a distal tip 13 in the ligamentum flavum (LF) tissue. Device 10 may comprise a cannula (or “needle”) and in some embodiments may include an elongate shaft 12 (including distal tip 13), a first actuator 14 for extending a cutting member 22 out of shaft 12, and a second actuator 16 for moving cutting member 22 along shaft 12 to cut tissue. In some embodiments, cutting member 22 may be coupled with an energy source 18 via one or more wires 20 or other connecting members. For example, in one embodiment cutting member 22 may comprise a radiofrequency (RF) cutting member, such as a bipolar or monopolar wire or wire loop, and power source 18 may comprise any suitable RF generator. Alternative embodiments are described further below.

With distal tip 13 located in ligamentum flavum tissue, and referring now to FIG. 2B, cutting member 22 may be extended out of a window or aperture on shaft 12. In one embodiment, as shown, cutting member 22 may be extended out of shaft 12 by advancing first actuator 14 along shaft 12. In alternative embodiments, actuator 14 may be moved or actuated in other ways to extend cutting member 22. In other alternative embodiments, cutting member 22 may automatically extend out of a window or aperture of shaft 12 when such a window or aperture is opened.

To confirm placement of distal tip 13 in ligamentum flavum (LF), any suitable technique may be used. For example, in some embodiments all or part of shaft 12 and distal tip 13 may be radiopaque, and a physician may view the location of shaft 12 and distal tip 13 via fluoroscopy. In some embodiments, cutting member 22 may also serve as a nerve stimulation member. In such embodiments, when cutting member 22 is extended into tissue, it may be activated, such as by transmitting RF energy, and the patient may be monitored for a response to the stimulation. For example, if cutting member 22 were accidentally placed into a nerve or nerve root, rather than ligamentum flavum (LF), activating cutting member 22 with a stimulating current would typically cause a response in the nerve, seen as a muscle twitch and/or detectable using a monitoring technique, such as electromyography (EMG). If cutting member 22 were in contact with a nerve, the physician might withdraw cutting member 22 and device 10 and reposition distal tip 13.

Once cutting member 22 is extended into ligamentum flavum (LF) tissue, energy, such as RF energy, may be transmitted to cutting member 22 via power source 18, and cutting member 22 may be moved through the tissue (hollow-tipped arrow), such as by sliding second actuator 16 along shaft 12. In some embodiments, as shown, cutting member 22 may be retracted, while in others it may be advanced, rotated, reciprocated or moved in any of a number of suitable ways to cut tissue.

As seen in FIG. 2C, one or more pieces of cut tissue 24 may be collected in shaft 12. For example, in one embodiment, suction may be applied at the proximal end of shaft 12, causing cut tissue 24 to be sucked into the hollow inner lumen of shaft 12. Alternatively, or additionally, cutting member 22 may have a configuration that directs cut tissue into shaft 12. In one embodiment, for example, cutting member 22 may comprise an electrosurgical RF wire loop configured to cut one or more strips of tissue, which pass beneath the wire as they are cut and pass into shaft 12. Cut tissue 24 may be removed from the patient by suctioning or otherwise pulling tissue 24 through shaft 12 and out its proximal end, by removing device 10 from the patient with tissue 24 contained in shaft 12, or some combination thereof.

After ligamentum flavum (LF) tissue on one side of the vertebra is removed, device 10 may be repositioned to remove similar tissue on the opposite side. As shown in FIG. 2D, device 10 may then be removed, leaving ligamentum flavum (LF) tissue reduced in size and no longer impinging on cauda equina (CE) or nerve root (NR) tissue. FIGS. 2A-2D demonstrate one embodiment of a method for removing tissue from a spine to treat spinal stenosis. A number of alternative embodiments are described below.

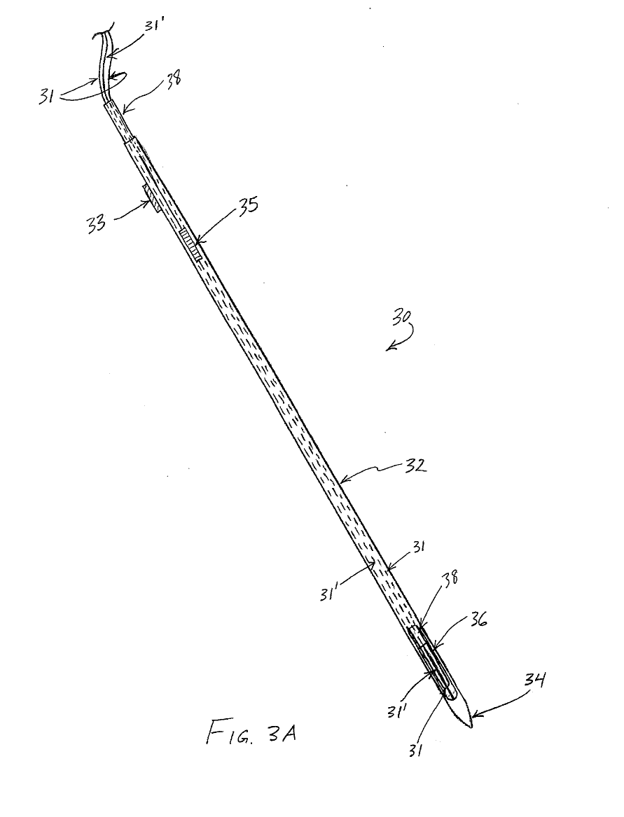

Referring now to FIGS. 3A and 3B, top and side/cross-sectional views, respectively, of one embodiment of a percutaneous tissue removal device 30 are shown. In this embodiment, device 30 may include a cannula/needle shaft 32 having a window 36 and a distal tip 34, a first actuator 33 for retracting a cover 38 over window 36, a second actuator 35 for retracting and advancing a cutting member 31 to cut tissue, and a return electrode 31′.

As best seen in FIG. 3B, cover 38 may comprise, in some embodiments, an inner shaft slidably disposed within the outer shaft 32. In embodiments using RF or other energy modalities, all or part of shaft 32 and/or cover 38 may be made of, coated with, covered with, mixed with or otherwise coupled with one or more insulating materials, to prevent damage to non-target tissues from heat, electricity or the like. Any suitable biocompatible insulating materials, either now known or hereafter invented or discovered may be used. In various embodiments, shaft 32 and cover 38 may have any suitable dimensions and may be made of any suitable materials. For example, in various embodiments, shaft 32 and cover 38 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. While device 30 of FIGS. 3A and 3B is shown having a rigid cannula shaft 32, in alternative embodiments, shaft 32 may be partially flexible and/or may have one or more articulating portions. Such alternative embodiments are described further below.

Cutting member 31 may comprise a wire loop RF electrode of a shape-memory or super-elastic material, such that when cover 38 is retracted to open window 36, the looped portion of cutting member 31 automatically extends out of window 36. Cutting member 31 may then be retracted, using second actuator 35, to cut tissue. Cutting member 31 may extend through shaft 32 (dotted lines) and exit proximally, for connection to an external power source (not shown), which may comprise any suitable RF source or other power source in alternative embodiments. In some embodiments, cutting member 31 and return electrode 31′ may form a bipolar electrosurgical cutting device, such that RF energy transmitted from a power source through cutting member 31 and thus through tissue is returned through device 30 via return electrode 31′. In an alternative embodiment, cutting member 31 may comprise a monopolar electrosurgical device, in which case a return electrode may be placed separately on a patient. Due to the proximity of nervous tissues, it may be advantageous to use bipolar electrosurgical devices in spinal procedures, although it may also be possible to use monopolar devices.

In an alternative embodiment, window 36 may be replaced with one or more small apertures, and first actuator 33 may be configured to extend cutting member 31 out of shaft 32 through such apertures and retract cutting member 31 back into shaft 32 after use. In such an embodiment, second actuator 35 may be used to move cutting member 31 back and forth longitudinally, relative to shaft 32, to cause cutting member 31 to cut tissue. In another alternative embodiment, cutting member 31 may be advanced out of one or more apertures on shaft 32, and shaft 32 may be retracted and/or advanced to move cutting member 31 through tissue and thus cut the tissue.

Cutting member 31 may comprise any suitable RF electrode, such as those commonly used and known in the electrosurgical arts. Any of a number of different ranges of radio frequency may be applied to cutting member 31, according to various embodiments. For example, some embodiments may use RF energy in a range of between about 70 hertz and about 5 megahertz. In some embodiments, the power range for RF energy may be between about 0.5 Watts and about 200 Watts. Additionally, in various embodiments, RF current may be delivered directly into conductive tissue or may be delivered to a conductive medium, such as saline or Lactate Ringers solution, which may in some embodiments be heated or vaporized or converted to plasma that in turn modifies target tissue. Similarly, cutting member 31 may be powered by an internal or external RF generator. Any suitable generators may be used, such as those commonly available at the present time and any generators invented hereafter. Examples of external generators that may be used include, but are not limited to, those provided by ValleyLabs (a division of Tyco Healthcare Group, LP (Pembroke, Bermuda and Princeton, N.J.)), Gyrus Medical, Inc. (Maple Grove, Minn.), and the high-frequency generators provided by Ellman International, Inc. (Oceanside, N.Y.).

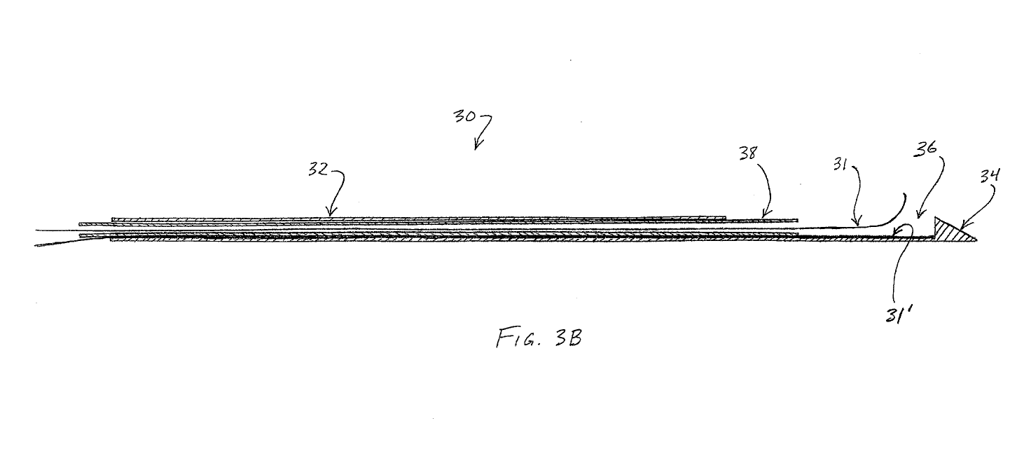

In various embodiments, many of which are described in further detail below, cutting member 31 may comprise one or more devices and may have any of a number of configurations, sizes, shapes and the like. In other words, although energy such as RF energy may be applied to a bipolar loop electrode cutting member 31, as shown in FIGS. 3 and 4, in alternative embodiments RF or other energy may be applied to any of a number of alternative tissue cutting devices. Examples of such cutting devices include, but are not limited to, blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices. Some embodiments may include an energy transmission member to cut tissue, while others may include a powered mechanical tissue cutter, a manual mechanical cutter, or some combination of energy transmitting, powered and/or mechanical cutters. For example, some embodiments may include one or more sharp blades coupled with an RF power source.

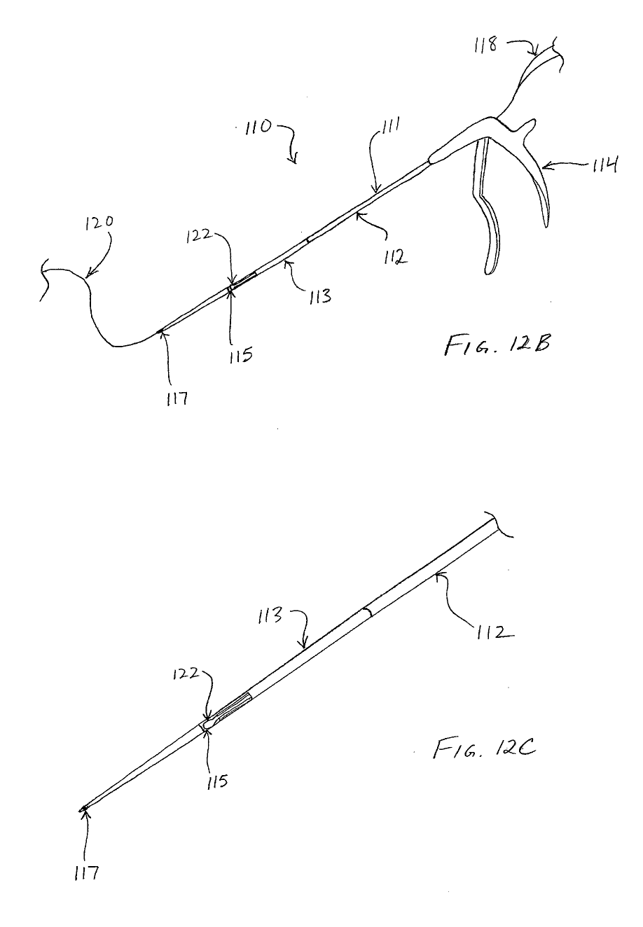

Referring now to FIGS. 4A-4E, a distal portion of percutaneous tissue removal device 30 is shown in greater detail. In FIG. 4A, the distal portion of device 30 is positioned in ligamentum flavum tissue 33, and cover 38 is in an advanced position, covering window 36. Window 36 may be covered, for example, as device 30 is passed into tissue. Cutting member 31 may be disposed in shaft 32 such that it is restrained by cover 38. In some embodiments, cutting member 31 may comprise a bipolar wire loop electrode, with only a distal loop portion of the wire exposed and with the proximal portions of the wire covered with insulating shafts 35 (not shown in FIGS. 3A and 3B), which may act to insulate the proximal portions of cutting member 31 and may also facilitate advancing and retracting cutting member 31 relative to shaft 32. In an alternative embodiment (e.g., FIG. 12D), cutting member may pass through one or more tracks or tubes coupled with an inner wall of shaft 32. An inner wall of cover 38 and/or shaft 32 may form a central lumen 39 of device 30, in which cut tissue may be collected and/or through which cut tissue may be removed.

Once the distal portion of device 30 is positioned in ligamentum flavum tissue 33, which may be confirmed, for example, by fluoroscopy, cover 38 may be retracted to open window 36, as in FIG. 4B. In some embodiments, when cover 38 is retracted, wire loop cutting member 31 may automatically extend through window 36 to contact tissue 33. In some embodiments, a stimulating current may then be passed through cutting member 31, and the patient may be monitored for nerve response, to ensure that cutting member 31 is not in contact with nerve tissue.

Cutting member 31 may then be activated, with current returning proximally through return electrode 31′. (In an alternative embodiment, cutting member 31 may be activated while window 36 is closed by cover 38, so that cutting member 31 is activated before it contacts tissue 33.) As in FIG. 4C, activated cutting member 31 may then be retracted to cut tissue 33. Cut tissue 33′ may then pass into lumen 39. In some embodiments, cutting member 31 may be shaped to urge cut tissue 33′ into lumen 39. Alternatively, or additionally, suction may be applied to lumen 39 to pull in cut tissue 33′.

In some embodiments, with one or more pieces of cut tissue 33′ in lumen 39, cover 38 may be advanced to close window 36, as in FIG. 4D. At this point, suction may be applied to lumen 39 (or continued, if already applied), to suck cut tissue 33′ through lumen 39 and out of the patient. In an alternative embodiment, cutting member 31 may be used to pull cut tissue 33′ through lumen. In another alternative embodiment, a separate tissue engaging member may coupled with cut tissue 33′ and be retracted to pull tissue 33′ through lumen 39. In yet another embodiment, device 30 may be removed from the patient with cut tissue 33′ trapped in lumen 39, cut tissue 33′ may be removed, and device 30 may optionally be reinserted into the patient to remove more tissue 33. In various embodiments, combinations of these methods for removing cut tissue 33′ from the patient may be used.

As shown in FIG. 4E, after cutting tissue 33, tissue cutting member 31 and cover 38 may be returned to their original positions. Optionally, device 30 may then be used to cut additional tissue 33.

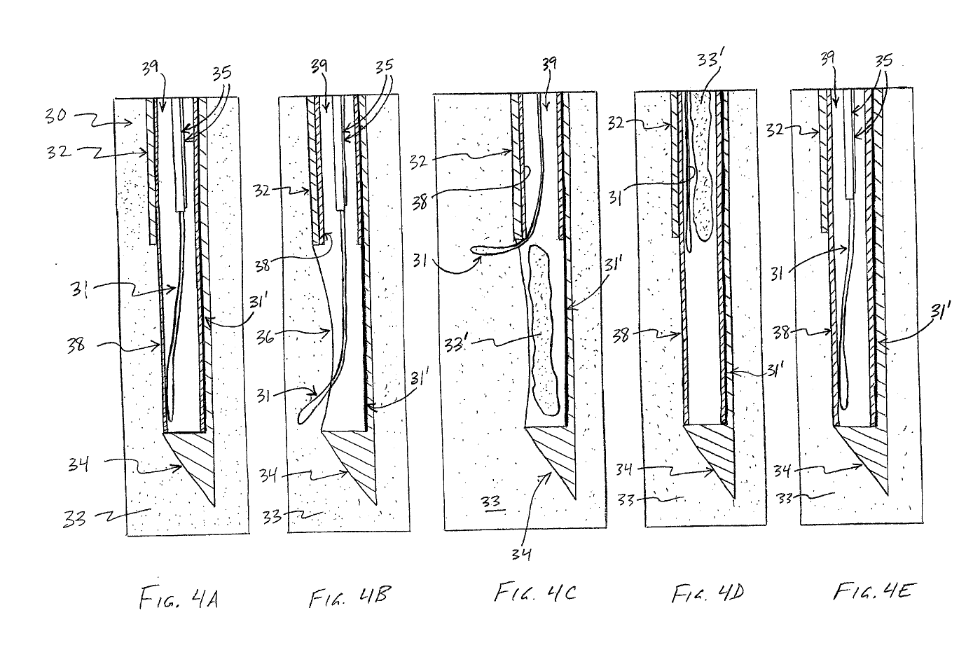

Referring now to FIGS. 5A-5E, in an alternative embodiment, a percutaneous tissue removal device 40 may include an outer shaft 42 having a distal tip 44 and a window 46, an inner shaft 47 slidably disposed in outer shaft 42 to act as a cover for window 46, and a blade shaft 48 slidably disposed in inner shaft 47 and including a pop-up blade 49 with a sharp blade edge 45. Outer shaft 42, inner shaft 47, blade shaft 48 and blade 49 may be made of any suitable materials, such as but not limited to the various metals, polymers, ceramics and composites listed above.

As shown in FIG. 5A, a distal portion of device 40 may be inserted into ligamentum flavum tissue 43, with inner shaft 47 advanced to close window 46 and to hold down blade 49. Inner shaft 47 may be retracted, as in FIG. 5B, to open window 46 and allow blade 49 to pop up, thus exposing blade edge 45 to tissue 43. In one embodiment, blade 49 may form a channel 50 below it when it pops up, thus creating a space through which cut tissue may pass into device 40.

As shown in FIG. 5C, once blade shaft 48 pops up into tissue, it may be retracted to cut tissue 43′, which passes through channel 50 into device 40. As shown in FIG. 5D, blade shaft 48 may then be advanced over cut tissue 43′, and cut tissue 43′ may be removed through lumen 41. In various embodiments, cut tissue 43′ may be removed from a patient by suctioning the tissue through lumen 41, by pulling the tissue through lumen 41 using a tissue engaging device, or by removing device 40 from the patient. As shown in FIG. 5E, blade shaft 48 may be retracted again, and may be advanced and retracted as many times as desired, to cause blade 49 to cut additional tissue 43″.

Referring to FIGS. 5F and 5G, more detailed side and bottom views, respectively, blade shaft 48 and blade 49 are provided. As seen in FIG. 5F, blade shaft 48 may comprise a hollow shaft, forming lumen 41. Pop-up blade 49 has cutting edge and forms channel 50 below it. In some embodiments, blade 49 may be made of a shape-memory or super-elastic material, which is compressible within inner shaft 47 and resumes its popped-up or “proud” configuration when released from constraint. FIG. 5G is a bottom view of blade shaft 48 and channel 50, from the perspective of the line A in FIG. 5F.

In alternative embodiments, a blade may be advanced rather than retracted, two blades may be moved toward one another, or other configurations of blades may be used. In some embodiments, energy (such as RF energy) may be transmitted to blade 49, to enhance tissue cutting. A number of different embodiments of bladed tissue cutting devices, any of which may be used percutaneously in various embodiments of the present invention, are described in U.S. patent application Ser. No. 11/405,848 (Original Attorney Docket No. 78117-200101), entitled “Mechanical Tissue Modificatino Devices and Methods,” and filed on Apr. 17, 2006, the full disclosure of which is hereby incorporated by reference.

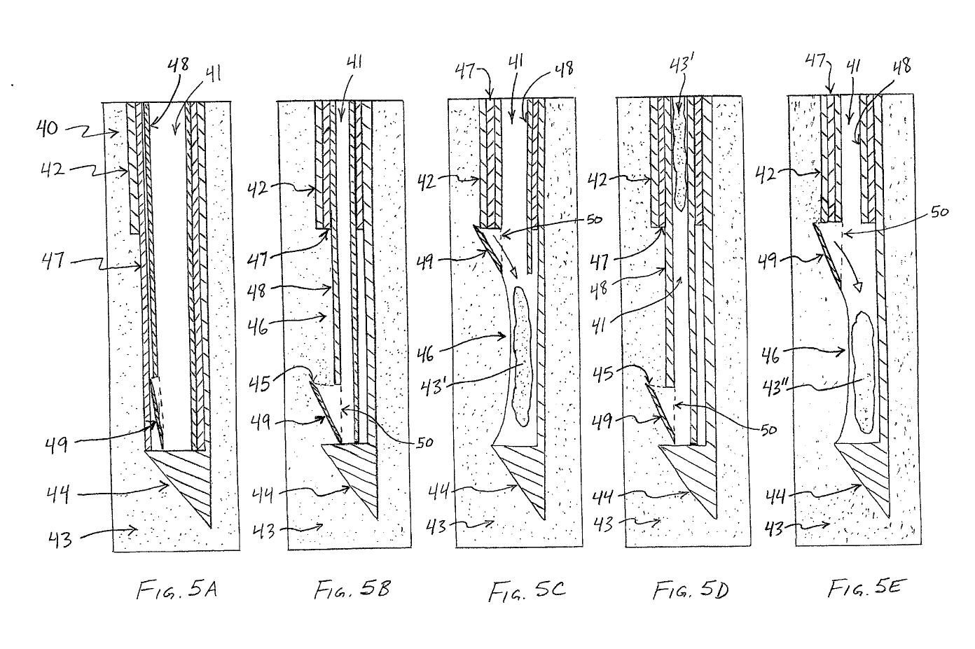

Referring now to FIGS. 6A-6E, in another alternative embodiment, a percutaneous tissue removal device 52 may include an outer shaft 54 forming a window 58, an inner shaft 60, a tissue engaging member 56 having multiple barbs 62, a first electrode 68 coupled with a lower surface of shaft 54, and a second electrode 69 coupled with an upper surface of shaft 54 (“upper side” being defined as the same side that window 58 opens on). Device 52 is similar to that described in U.S. patent application Ser. No. 11/193,581, by Solsberg et al., entitled “Spinal Ligament Modification,” the full disclosure of which is hereby incorporated by reference. Device 52, however, includes additional features not described in the foregoing reference.

During percutaneous insertion of device 52 into ligamentum flavum tissue 66, inner shaft 60 may be in an advanced position to close window 58. In some embodiments, window 58 may be visible under external imaging guidance, such as fluoroscopy, to facilitate orienting window 58 away from the epidural space of the spine and thus protect non-target structures from injury during the surgical procedure. In other embodiments, an endoscopic visualization device may be coupled with device 52 to facilitate internal imaging. Examples of such visualization devices include, but are not limited to, flexible fiber optic scopes, CCD (charge-coupled device) or CMOS (complementary metal-oxide semiconductor) chips at the distal end of flexible probes, LED illumination, fibers or transmission of an external light source for illumination, and the like.

Once a distal portion of device 52 is positioned in the ligamentum flavum or other tissue removal site, nerve stimulating energy may be transmitted through first electrode 68 or second electrode 69, and the patient may be monitored for a nerve response. If a nerve response is detected, it may be determined that device 52 is too close to nervous tissue to safely perform a procedure, and device 52 may be repositioned in tissue 66. Optionally, the other electrode, which was not already activated, may be activated to see if it stimulates nervous tissue. Alternative embodiments may include only one electrode or more than two electrodes. In any case, based on the stimulation or lack of stimulation of nerve tissue by one or both electrodes 68, 69, it may be determined that device 52 is in a safe location for performing a tissue removal procedure. Various methods and apparatus for stimulating electrodes and monitoring for response are described in U.S. patent application Ser. No. 11/429,377 (Attorney Docket No. 026445-000724US), entitled “Spinal Access and Neural Localization,” and filed Jul. 13, 2006, the full disclosure of which is hereby incorporated by reference.

With the distal portion of device 52 positioned in a desired location in ligamentum flavum tissue 66, inner shaft 60 may be retracted/slid proximally so that it no longer closes window 58, as shown in FIG. 6B. If it was not already present in device 52, tissue engaging member 56 may be inserted through inner shaft 60 so that it contacts ligamentum flavum tissue 66 via window 58. In various embodiments, tissue engaging member 56 may comprise a needle, hook, blade, tooth or the like, and may have at least one flexible barb 62 or hook attached to its shaft. In some embodiments, barbs 62 may extend around approximately 120 degrees of the circumference of the shaft. In some embodiments, barbs 62 may be directed towards the proximal end of the tool, as in FIGS. 6A-6E. When tissue engaging member 56 is retracted slightly, barbs 62 engage a segment of tissue 66. Depending on the configuration of barbs 62, the tissue sample engaged by barbs 62 may be generally cylindrical or approximately hemispherical.

Referring to FIG. 6C, once tissue engaging member 56 has engaged the desired tissue 66, inner shaft 60, which is preferably provided with a sharpened distal edge, is advanced so that it cuts the engaged tissue section 66′ or sample loose from the surrounding tissue 66. Hence, inner shaft 60 also functions as a cutting means in this embodiment. In alternative embodiments, a cylindrical outer cutting element may be extended over outer shaft 52 to cut tissue 66.

Referring to FIG. 6D, once tissue 66′ has been cut, tissue engaging member 56 may be pulled back through inner shaft 60 so that cut tissue segment 66′ may be retrieved and removed from barbs 62. Tissue engaging member 56 may then be advanced, as in FIG. 6E, and the process of engaging and cutting tissue may be repeated until a desired amount of ligamentum flavum tissue 66 has be removed (e.g., when a desired of amount of decompression has been achieved).

In various embodiments, device 52 may have one or more additional features, some of which are described in greater detail below. For example, in some embodiments, the distal portion of device 52 may be articulatable relative to a proximal portion of device 52, to facilitate passage of the distal portion into or through curved passages or channels, such as an intervertebral foramen. In another embodiment, the distal portion of device 52 may be flexible and/or curved, again to facilitate passage at least partway into an intervertebral foramen. In either an articulatable or a flexible embodiment, device 52 may optionally also include a guidewire coupling member for attaching device 52 with a guidewire. Such a guidewire may be used to pull device 52 into place and apply force to device 52 to urge barbs 62 into tissue 66. Examples of various guidewire mechanisms are described in greater detail in U.S. patent application Ser. Nos. 11/468,247 and 11/468,252 (Attorney Docket Nos. 026445-001000US and 026445-001100US, respectively), both of which are entitled “Tissue Access Guidewire System and Method, and both of which were filed on Aug. 29, 2006, the full disclosures of which are hereby incorporated by reference. In an alternative embodiment, device 52 may include a guidewire lumen or track over so that device 52 may be passed into the spine over a guidewire. Some of these optional features are described in greater detail below.

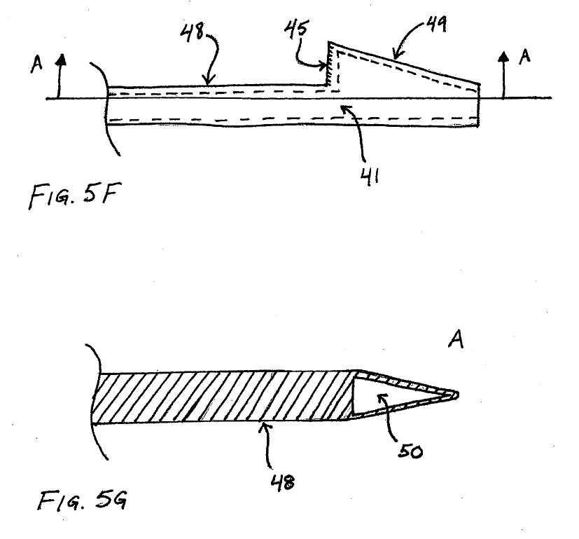

Referring now to FIG. 7, in another alternative embodiment, a percutaneous tissue removal device 130 may include a shaft 132 having a window 134 therein, a cover 136 or inner shaft slidably disposed in shaft 132 for opening and closing window 134, and a cylindrical, rotating blade 138 having a sharpened blade edge 139 and a hollow central channel 137. Device 130 may be coupled proximally with a drive mechanism and power source (not shown) to drive blade 138. As in previously described embodiments, cover 136 may retract to expose blade 138. Blade 138 may rotate (curved arrows) as well as advance and retract (double, hollow-tipped arrow) to cut tissue, which may then pass through hollow channel 137. In some embodiments, device 130 may include or be couplable with a suction device to suck cut tissue through channel 137. Blade 138 may be made of metal or any other suitable material, such as polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Ceramics may include but are not limited to aluminas, zirconias, and carbides.

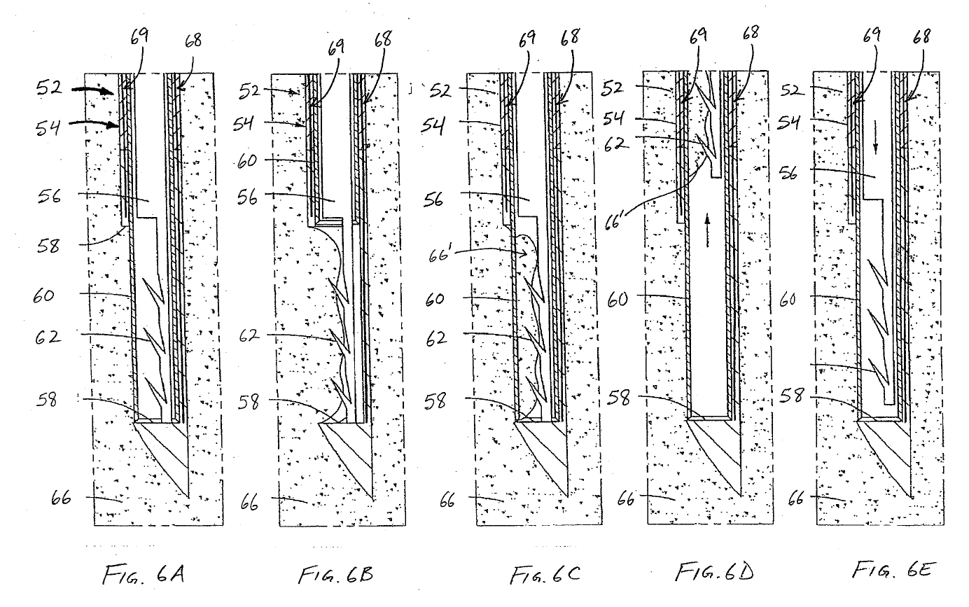

Referring to FIG. 8, in one embodiment, a percutaneous tissue removal device 140 may include a shaft 142 having a window 144 therein, a cover 146 or inner shaft slidably disposed in shaft 142 and forming a lumen 145, and a cylindrical, rotating blade 148 having a sharpened blade edge 149 and coupled with a drive shaft 147. Drive shaft 147 may be coupled proximally with a drive mechanism and power source (not shown) to drive blade 148. Blade 148 may rotate (curved arrows) as well as advance and retract (double, hollow-tipped arrow) to cut tissue, which may then pass through blade 148 and into lumen 145. In some embodiments, device 140 may include or be couplable with a suction device to suck cut tissue through lumen 145. Blade 148 may be made of metal or any other suitable material, such as polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Ceramics may include but are not limited to aluminas, zirconias, and carbides.

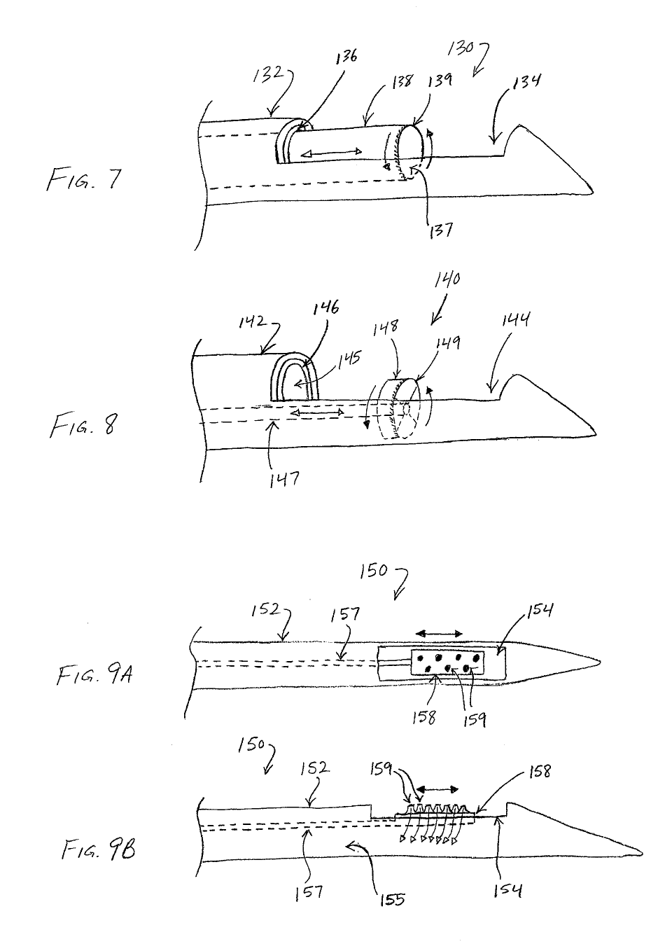

Referring now to FIGS. 9A and 9B, in one embodiment, a percutaneous tissue removal device 150 may include a shaft 152 having a window 154 therein forming a lumen 155, and a reciprocating tissue cutter 158 having multiple tissue cutting elements 159 and being attached to a drive shaft 157. Optionally, device 150 may also include a cover as described in various embodiments above but not shown in FIGS. 9A and 9B. Drive shaft 157 may be coupled proximally with a drive mechanism and power source (not shown) to drive reciprocating tissue cutter 158. Tissue cutter 158 may reciprocate (double, solid-tipped arrow) to cause cutting elements 159 to cut tissue, which may then pass through cutting elements 159 and into lumen 155. In some embodiments, device 150 may include or be couplable with a suction device to suck cut tissue through lumen 155. Tissue cutter 158 may have any suitable number, shape and size of cutting elements 159, and both cutter 158 and elements 159 may be made of metal or any other suitable material, such as polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Ceramics may include but are not limited to aluminas, zirconias, and carbides.

Any of a number of suitable powered tissue removal devices may be used percutaneously to remove ligamentum flavum tissue and/or bone in the spine to treat neural impingement, neurovascular impingement and/or spinal stenosis. Examples of various alternative powered tissue removal devices are provided in U.S. patent application Ser. No. 11/406,486 (Original Attorney Docket No. 78117-200501), entitled “Powered Tissue Modification Devices and Methods,” and filed Apr. 17, 2006, the full disclosure of which is hereby incorporated by reference. Other powered devices which may be used percutaneously are described in U.S. patent application Ser. Nos. 11/468,247 and 11/468,252, both of which were previously incorporated by reference.

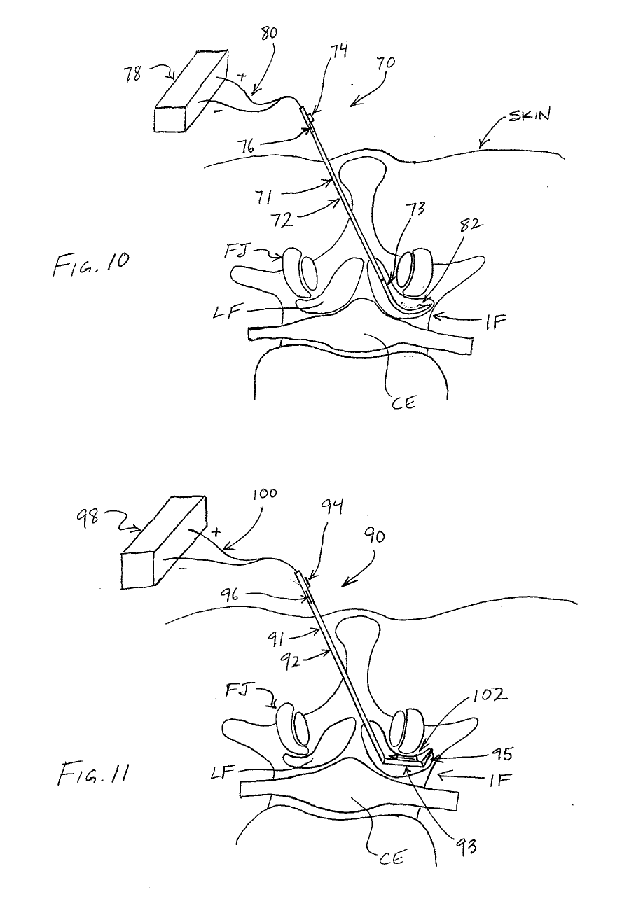

Referring now to FIG. 10, in one embodiment, a percutaneous tissue removal device 70 may include a cannula/needle shaft 71 having a rigid proximal portion 72 and a flexible distal portion 73. Device 70 may also include an energy transmitting cutting member 82, a first actuator 74 for bending distal portion 73, a second actuator 76 for moving cutting member 82 along distal portion 73, and a power source 78 coupled with cutting member 82 via wires 80. In some embodiments, distal portion 73 may be sufficiently rigid to penetrate a patient's soft tissue and ligamentum flavum (LF) but also sufficiently flexible to be able to bend or articulate relative to proximal portion 72. In various embodiments, any of a number of actuating/flexing/bending mechanisms may be incorporated in device 70 to allow distal portion 73 to flex, such as pull wires, push wires or the like. Examples and further description of articulating tissue cutting devices are provided, for example, in U.S. patent application Ser. No. 11/538,345 (Attorney Docket No. 026445-001300US), entitled “Articulating Tissue Cutting Devices,” and filed Oct. 3, 2006, the full disclosure of which is hereby incorporated by reference.

In various alternative embodiments, device 70 may be percutaneously advanced into a patient to advance distal portion 73 in ligamentum flavum tissue, between ligamentum flavum tissue and bone, and between ligamentum flavum tissue and nervous tissue. Flexible distal portion 73 may allow or facilitate passage of at least part of distal portion 73 into an intervertebral foramen (IF) of the spine. Cutting member 82 and the various other features of device 70 may be similar to any of those described in reference to alternative embodiments above.

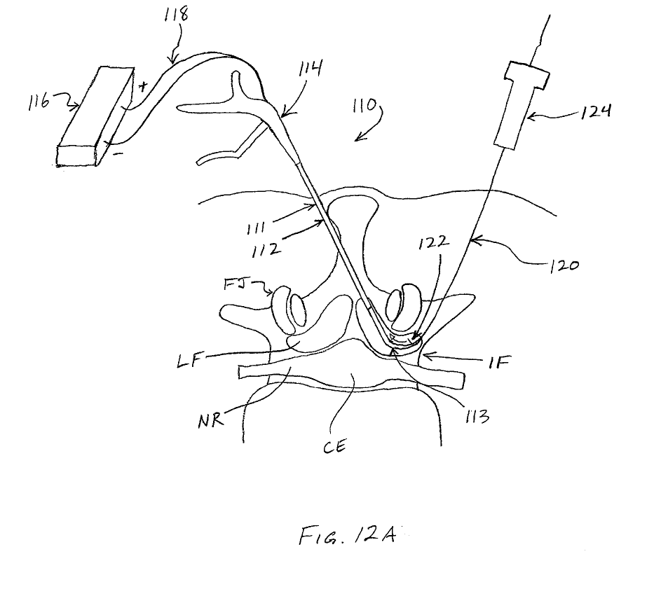

Referring now to FIG. 11, in an alternative embodiment, a percutaneous tissue removal device 90 may include a cannula/needle shaft 91 having a rigid proximal portion 92, a rigid distal portion 93 that articulates relative to proximal portion 92, and a distal tip 95 that articulates relative to distal portion 93. Device 90 may also include an energy transmitting cutting member 102, a first actuator 94 for articulating distal portion 93 and distal tip 95, a second actuator 96 for moving cutting member 102 along distal portion 93, and a power source 98 coupled with cutting member 102 via wires 100. As with the previously described embodiment, any of a number of actuating mechanisms may be incorporated in device 90 for actuation of distal portion 93 and distal tip 95, such as but not limited to those described in U.S. patent application Ser. No. 11/538,345, which was previously incorporated by reference. Cutting member 102 and the various other features of device 90 may be similar to any of those described in reference to alternative embodiments above.

Referring now to FIG. 12A, another embodiment of a percutaneous tissue removal device 110 is shown in place for performing a procedure in a patient. In one embodiment, tissue removal device 110 may include a shaft 111 having a rigid proximal portion 112, a flexible distal portion 113, an energy transmitting cutting member 122, a handle 114 coupled with shaft proximal end 112 for articulating and moving cutting member 122 along distal portion 113, and a power source 116 coupled with cutting member 122 via wires 118. Additionally, device 110 may include a guidewire 120, which is couplable with distal portion 113, and a guidewire handle 124 removably couplable with guidewire 120. Guidewire 120 and guidewire handle 124 may be used to pull distal portion 113 into a desired location in the patient. Such a method and system are described in greater detail in U.S. patent application Ser. Nos. 11/468,247 and 11/468,252, which were previously incorporated by reference.

As seen in FIGS. 12B and 12C, distal shaft portion 113 may include a window 115, through which a wire loop electrode cutting member 122 may extend or simply be exposed. Distal portion 113 may also include a guidewire coupling member 117 at or near its extreme distal end. Again, for further details regarding various guidewire coupling members 117 and corresponding guidewires, reference may be made to U.S. patent application Ser. Nos. 11/468,247 and 11/468,252.

FIG. 12D shows the mechanism of cutting member 122 in greater detail. A similar mechanism is described in U.S. patent application Ser. No. 11/375,265 (Original Attorney Docket No. 78117-375,265), entitled “Methods and Apparatus for Tissue Modification,” and filed Mar. 13, 2006, the full disclosure of which is hereby incorporated by reference. Wire loop electrode cutting member 122 may comprise any suitable RF electrode, such as those commonly used and known in the electrosurgical arts, and may be powered by an internal or external RF generator, such as the RF generators provided by ValleyLabs (a division of Tyco Healthcare Group, LP (Pembroke, Bermuda and Princeton, N.J.)), Gyrus Medical, Inc. (Maple Grove, Minn.), and the high-frequency generators provided by Ellman International, Inc. (Oceanside, N.Y.). Any of a number of different ranges of radio frequency may be used, according to various embodiments. For example, some embodiments may use RF energy in a range of between about 70 hertz and about 5 megahertz. In some embodiments, the power range for RF energy may be between about 0.5 Watts and about 200 Watts. Additionally, in various embodiments, RF current may be delivered directly into conductive tissue or may be delivered to a conductive medium, such as saline or Lactate Ringers solution, which may in some embodiments be heated or vaporized or converted to plasma that in turn modifies target tissue.

In some embodiments, cutting member 122 may be caused to extend out of window 115, expand, retract, translate and/or the like. Some embodiments may optionally include a second actuator (not shown), such as a foot switch for activating an RF generator to delivery RF current to an electrode.

Insulators 126 may be disposed around a portion of wire loop cutting member 122 so that only a desired portion of cutting member 122 may transfer RF current into target tissue. Cutting member 122, covered with insulators 126 may extend proximally into support tubes 124. In various alternative embodiments, cutting member 122 may be bipolar or monopolar. For example, as shown in FIG. 12D, a sleeve 128 housed toward the distal portion of window 115 may act as a return electrode for cutting member 122 in a bipolar device. Cutting member 122 may be made from various conductive metals such as stainless steel alloys, nickel titanium alloys, titanium alloys, tungsten alloys and the like. Insulators 126 may be made from a thermally and electrically stable polymer, such as polyimide, polyetheretherketone (PEEK), polytetrafluoroethylene (PTFE), polyamide-imide, or the like, and may optionally be fiber reinforced or contain a braid for additional stiffness and strength. In alternative embodiments, insulators 126 may be composed of a ceramic-based material. Distal shaft portion 113 may also be made of or coated or covered with one or more insulating materials, such as those just listed.

In one embodiment, cutting member 122 may be housed within distal portion 113 during delivery of distal portion 113 into a patient, and then caused to extend up out of window 115, relative to the rest of distal portion 113, to remove tissue. Cutting member 122 may also be flexible so that it may pop or bow up out of window 115 and may deflect when it encounters hard tissue surfaces. Cutting member 122 may have any of a number of shapes, such as curved, flat, spiral or ridged. Cutting member 122 may have a diameter similar to the width of distal portion 113, while in alternative embodiments it may expand when extended out of window 115 to have a smaller or larger diameter than that of distal portion 113. Pull wires (not shown) may be retracted proximally, in a manner similar to that described above, in order to collapse cutting member 122, decrease the diameter and lower the profile of the cutting member 122, and/or pull cutting member 122 proximally to remove tissue or be housed within distal portion 113. The low profile of the collapsed cutting member 122 facilitates insertion and removal of distal portion 113 into and out of a patient prior to and after tissue modification. As the cutting member 122 diameter is reduced, support tubes 124 deflect toward the center of distal portion 113.

In an alternative embodiment (not shown), tissue modification device 110 may include multiple RF wire loops or other RF members. In another embodiment, device 110 may include one or more blades as well as an RF wire loop. In such an embodiment, the wire loop may be used to remove or otherwise modify soft tissues, such as ligamentum flavum, or to provide hemostasis, and blades may be used to modify hard tissues, such as bone. In other embodiments, as described further below, two separate tissue modification devices 110 (or more than two devices) may be used in one procedure to modify different types of tissue, enhance modification of one type of tissue or the like.

In other alternative embodiments, tissue modification devices 110 may include tissue modifying members such as a rongeur, a curette, a scalpel, a scissors, a forceps, a probe, a rasp, a file, an abrasive element, one or more small planes, a rotary powered mechanical shaver, a reciprocating powered mechanical shaver, a powered mechanical burr, a laser, an ultrasound crystal a cryogenic probe, a pressurized water jet, a drug dispensing element, a needle, a needle electrode, or some combination thereof. In some embodiments, for example, it may be advantageous to have one or more tissue modifying members that stabilize target tissue, such as by grasping the tissue or using tissue restraints such as barbs, hooks, compressive members or the like. In one embodiment, soft tissue may be stabilized by applying a contained, low-temperature substance (for example, in the cryo-range of temperatures) that hardens the tissue, thus facilitating resection of the tissue by a blade, rasp or other device. In another embodiment, one or more stiffening substances or members may be applied to tissue, such as bioabsorbable rods. In various embodiments, energy such as RF energy may be transmitted to any or all such tissue modification members, such as an RF transmitting blade or the like.

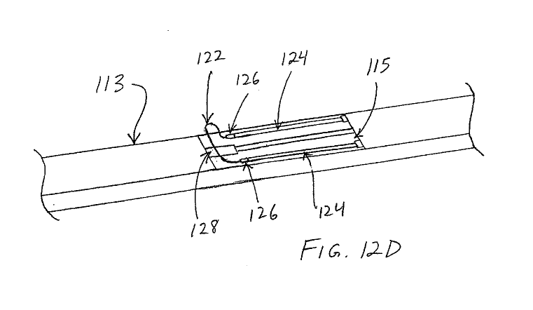

Referring now to FIG. 13, in another embodiment a percutaneous tissue removal device 210 may comprise a multi-wire, partially flexible rongeur-like device. Such devices are described in greater detail in U.S. patent application Ser. No. 11/535,000 (Attorney Docket No. 026445-000910US), titled “Tissue Cutting Devices and Methods,” and filed on Sep. 25, 2006, the full disclosure of which is hereby incorporated by reference. In one embodiment, device 210 may include a shaft 211 having a proximal portion 212 and a distal portion 213. In some embodiments, proximal shaft portion 212 is predominantly rigid, and at least part of distal shaft portion 213 is flexible. Proximal shaft portion 212 may be coupled with or may extend from a proximal handle 216. At least two flexible wires may slidably extend through a portion of proximal shaft portion 212 and distal shaft portion 213 so that their distal ends attach to a proximal blade 226 and so that they can advance proximal blade toward a distal blade 226 to cut tissue between them. A guidewire connector 230 may be coupled with distal shaft portion 213 anywhere along it length, such as at or near its extreme distal end. In some embodiments, tissue cutter device 210 (or a system including device 210) may further include additional features, such as a guidewire 232 with a sharp distal tip 233 and configured to couple with guidewire connector 230, and a distal handle 234 (or “guidewire handle”) with a tightening lever 236 for coupling with guidewire 232.

In some embodiments, tissue cutter device 210 may be advanced percutaneously into a patient's back by coupling guidewire connector 230 with guidewire 232 that has been advanced between target and non-target tissues, and then pulling guidewire 232 to pull device 210 between the tissues. In alternative embodiments, device 210 may be advanced over guidewire 232, such as via a guidewire lumen or track. The flexibility of distal shaft portion 213 may facilitate passage of device 210 between tissues in hard-to-reach or tortuous areas of the body, such as between a nerve root (NR) and facet joint and through an intervertebral foramen (IF). Generally, device 210 may be advanced to a position such that blades 226 face tissue to be cut in a tissue removal procedure (“target tissue”) and one or more non-cutting surfaces of device 210 face non-target tissue, such as nerve and/or neurovascular tissue. In the embodiment shown in FIG. 13, blades 226 are positioned to cut ligamentum flavum (LF) and may also cut hypertrophied bone of the facet joint, such as the superior articular process (SAP). (Other anatomical structures depicted in FIG. 13 include the vertebra (V) and cauda equina (CE)).

Before or after tissue cutter device 210 is pulled into the patient to pull blades 226 to a desired position, guidewire 232 may be removably coupled with distal handle 234, such as by passing guidewire 232 through a central bore in handle 234 and tightening handle 234 around guidewire 232 via a tightening lever 236. Proximal handle 216 and distal handle 234 may then be pulled (hollow-tipped arrows) to apply tensioning force to device 210 and thus to urge the cutting portion of device 210 (e.g., blades 226) against ligamentum flavum (LF), superior articular process (SAP), and/or other tissue to be cut. Proximal handle 216 may then be actuated, such as by squeezing in the embodiment shown, which advances the flexible wires and proximal blade 226, to cut tissue between blades 226. Proximal handle 216 may be released and squeezed as many times as desired to remove a desired amount of tissue. When a desired amount of tissue has been cut, guidewire 232 may be released from distal handle 234, and cutter device 210 and guidewire 232 may be removed from the patient's back.

In various alternative embodiments of the method just described, device 210 may be positioned with at least part of distal shaft portion 213 located in ligamentum flavum tissue or above ligamentum flavum in contact with bone. In the latter example, device 210 may be use to cut bone while leaving the ligamentum flavum largely or entirely intact. Again, for further description of various mechanical tissue modification devices, any of which may be used percutaneously, reference may be made to U.S. patent application Ser. No. 11/535,000, which was previously incorporated by reference.

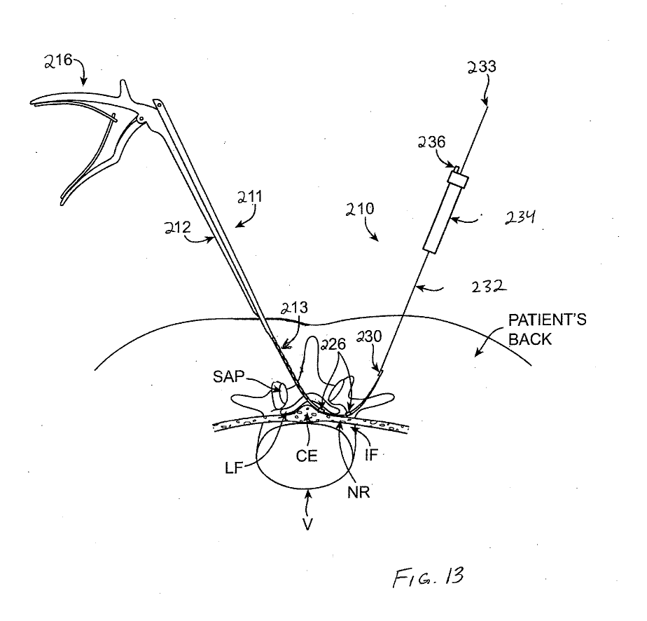

Referring now to FIG. 14, in some embodiments, a percutaneous tissue access device 306 may be used to provide a safe conduit for inserting and using one or more tissue modification devices to treat spinal stenosis or neural/neurovascular impingement. Examples of access device 306 are described in greater detail in U.S. patent application Ser. Nos. 11/468,247 and 11/468,252, which were previously incorporated by reference. In some embodiments, tissue access device 360 may be percutaneously advanced to a position in a patient's back using guidewire system 240.

Tissue access device 306 may include, for example, a proximal handle 307 having a hollow bore 308 and an actuator 309, a hollow shaft 310 extending from proximal handle 307 and having a distal curved portion and a distal window 312, and a guidewire coupling member 314 coupled with a tapered distal end of shaft 310. Any of a number of different tissue modification devices 316, 317, 320 may be inserted and removed from access device 306 to perform a tissue modification procedure, such as a rongeur 316, an ultrasound device 317 (including a wire 318 and generator 319), and an abrasive device 320. Handle 307 and actuator 309 may be used to activate one or more tissue modifying members of various tissue modification devices. For example, rongeur 316 may be advanced into hollow bore 308 and shaft 310, to position blades 321 of rongeur 316 so as to be exposed through window 312, and to lock a locking member 315 of rongeur 316 within handle 307. Actuator 309 may then be moved back and forth (by squeezing and releasing, in the embodiment shown) to move one or both blades 321 back and forth to cut target tissue. Optionally, rongeur 316 may then be removed from access device 306 and a different modification device 317, 320 inserted to further modify target tissue. Actuator 309 may be used with some modification devices and not others. Again, in some embodiments, access device 306, guidewire system 240 and one or more modification devices 316, 317, 320 may be provided as a system or kit.

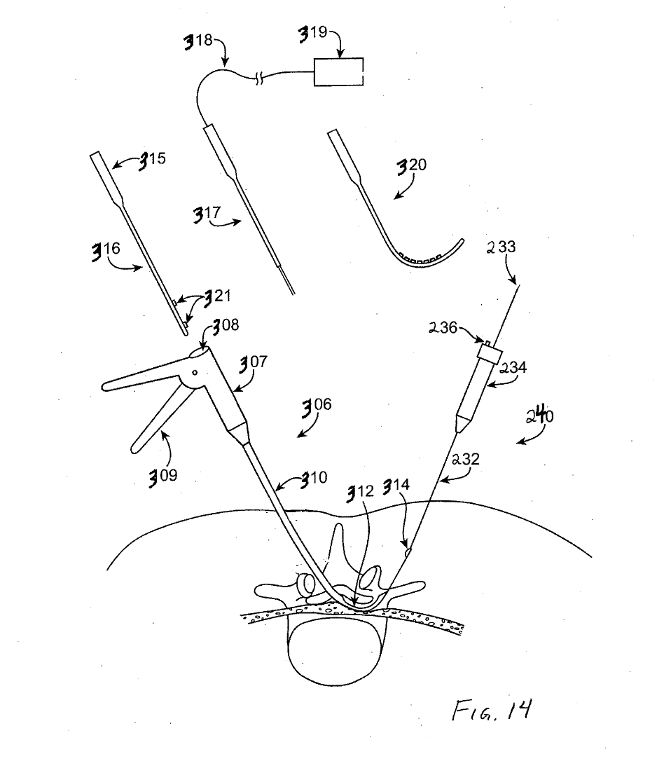

Referring now to FIGS. 15A-15E, in an alternative embodiment, a shield or barrier 500 (which may alternatively or additionally comprise a tissue capture device) may be positioned between target and non-target tissue in a patient before the target tissue is modified. Such barriers 500 may be slidably coupled with, fixedly coupled with, or separate from the tissue modification devices with which they are used. In various embodiments, a barrier may be delivered between target and non-target tissues before delivering the tissue modification device, may be delivered along with the tissue modification device, or may be delivered after delivery of the tissue modification device but before the device is activated or otherwise used to modify target tissue. Generally, such a barrier may be interposed between the non-target tissue and one or more tissue modification devices to prevent unwanted damage of the non-target tissue. Detailed description of various embodiments of barrier devices is provided in U.S. patent application Ser. No. 11/405,859 (Original Attorney Docket No. 78117-200601), titled “Tissue Modification Barrier Devices and Methods,” and filed Apr. 17, 2006, the full disclosure of which is hereby incorporated by reference.

FIG. 15A shows a distal portion of an introducer device 514 through which barrier 500 may be introduced. FIGS. 15B and 15C show one embodiment of barrier 500 partially deployed and in cross-section, respectively. Typically, barrier 500 will have a first, small-profile configuration for delivery to an area near non-target tissue and a second, expanded configuration for protecting the non target tissue. In various embodiments, barrier 500 may have any of a number of sizes and shapes. For example, barrier 500 is shown in FIG. 15B with a tapered end. In an alternative embodiment, barrier 500 may instead have a squared-off end, a more rounded end, or the like.

In various embodiments, barrier 500 may be configured as one piece of super-elastic or shape-memory material, as a scaffold with material draped between the scaffolding, as a series of expandable wires or tubes, as a semicircular stent-like device, as one or more expandable balloons or bladders, as a fan or spring-loaded device, or as any of a number of different devices configured to expand upon release from delivery device 514 to protect tissue. As shown in FIGS. 15B and 15C, barrier 500 may comprise a sheet of material disposed with a first end 502a abutting a second end 502b within introducer device 514 and unfurling upon delivery.

In an alternative embodiment, as shown in FIGS. 15D and 15E, opposite ends 522a and 522b of a barrier 520 may overlap in introducer device 514. Generally, barrier 500, 520 may be introduced via introducer device 514 in one embodiment or, alternatively, may be introduced via any of the various means described above for introducing a tissue modification device. In some embodiments, barrier 500, 520 may be fixedly coupled with or an extension of a tissue modification device. Barrier 500, 520 may also include one or more lumens, rails, passages, guidewire coupling members or the like for passing or connecting with a guidewire or other guide member, for introducing, removing, steering, repositioning, or exchanging any of a variety of tissue modification, drug delivery, or diagnostic devices, for passing a visualization device, for passing a device designed for neural localization, for providing irrigation fluid and/or suction at the tissue modification site, and/or the like. In some embodiments, barrier 500, 520 is advanced over multiple guidewires and the guidewires remain in place during a tissue modification procedure to enhance the stability and/or maintain positioning of barrier 500, 520.

Introducer device 514 may comprise any suitable catheter, introducer, sheath or other device for delivering one or more barrier devices into a patient. In various alternative embodiments, barrier devices may be delivered into a patient either through a delivery device, over one or more guide members, behind one or more guidewires, or some combination thereof. In various embodiments, introducer device 514 may have any suitable dimensions, profile or configuration. For example, in various embodiments, introducer device 514 may have a circular cross-sectional shape, an oval cross-sectional shape, or a shape that varies between circular and oval along the length of device 514. In some embodiments, an outer diameter of introducer device 514 or delivery device 601 may range from about 0.025″ to about 1.0″, with a wall thickness range of about 0.001″ to about 0.125″. Optionally, introducer device 514 may taper along its length. Introducer device 514 may be rigid, partially flexible or flexible along its entire length and may be made from any suitable material, such as but not limited to: a metal, such as stainless steel (303, 304, 316, 316L), nickel-titanium alloy, cobalt-chromium, or nickel-cobalt; a polymer, such as nylon, silicone, polyetheretherketone (PEEK), polyetherketoneketone (PEKK), polytetrafluoroethylene (PTFE), polyurethane (Tecothane), Pebax (co, USA), polycarbonate, Delrin (co, USA), high-density polyethylene (HDPE), low-density polyethylene (LDPE), HMWPE, and UHMWPE; or a combination of metals and polymers. Introducer device 514 may be manufactured by methods known in the art, such as CNC machining, extruding, casting, injection molding, welding, RF shaping, electrochemical fabrication (EFAB), LIGA (lithographic, galvanoforming and abforming), electrical discharge machining (EDM) laser machining, silicon micromachining, weaving, braiding or non-woven fabrication techniques (e.g., spunbound, meltblown, and the like). In some embodiments, introducer device 514 may be woven from polymer or metal into a tube-like structure for flexibility and conformability. Such embodiments may optionally be fiber-reinforced for added strength to allow for a thinner wall thickness.

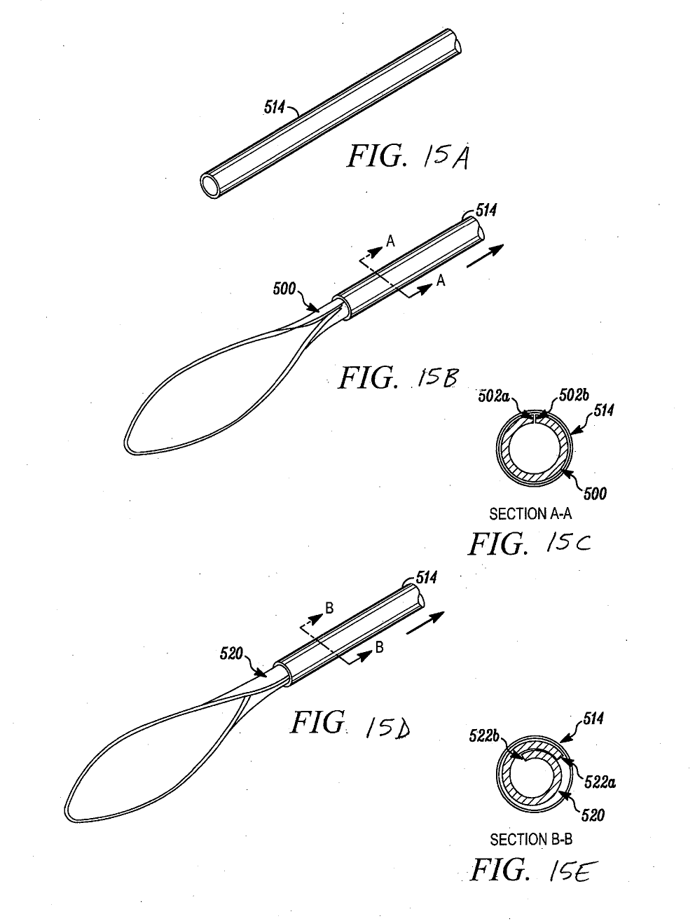

FIGS. 16A and 16B illustrate how, in one embodiment, a barrier device 1020 extending through a delivery device 601 may help protect tissue during a tissue modification procedure involving use of a tissue modification device 1024. In various embodiments, tissue modification device 1024 may include, but is not limited to, a rongeur, a curette, a scalpel, one or more cutting blades, a scissors, a forceps, a probe, a rasp, a file, an abrasive element, one or more small planes, an electrosurgical device, a bipolar electrode, a unipolar electrode, a thermal electrode a rotary powered mechanical shaver, a reciprocating powered mechanical shaver, a powered mechanical burr, a laser, an ultrasound crystal, a cryogenic probe, a pressurized water jet, or any combination of such devices. Tissue modification device 1024 may be advanced and retracted (double-headed arrows) freely on one side of barrier device 1020 and may be used to modify tissue, while barrier device 1020 protects non-target tissue from sustaining unwanted damage. In some embodiments, barrier device 1020 may also be used to help guide tissue modification device 1024 to and/or from a position for performing a tissue modification procedure. Such guidance may be achieved by a shape, surface characteristic and/or one or more guide features of barrier device 1020, according to various embodiments.

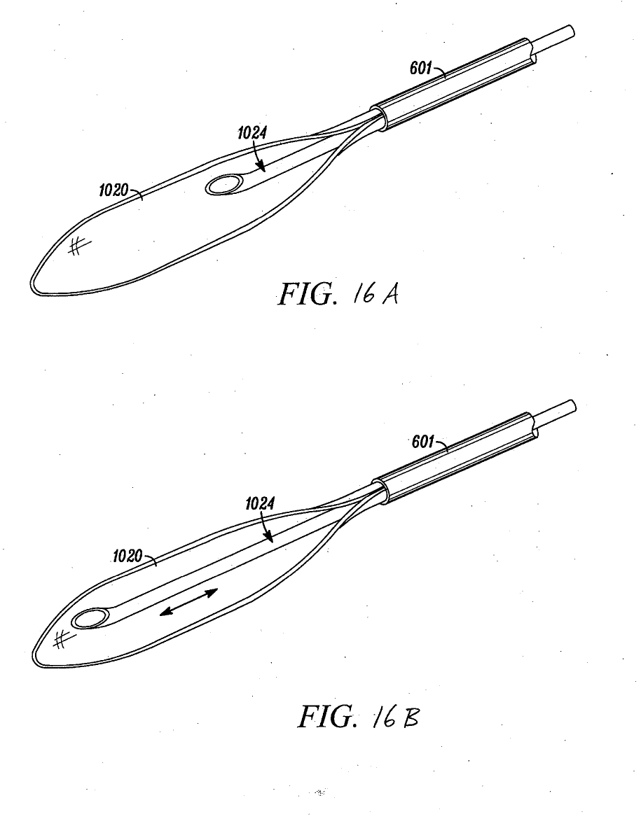

Turning to FIGS. 17A and 17B, in another embodiment, a barrier device 1030 may include an open, shape-changing portion 1030, closed, elongate extensions 1034 extending from either end of shape-changing portion 1030, and at least one guide feature 1035 extending through its length. Guide feature 1035 may include, in various embodiments, one or more guidewires (as shown), rails, impressions, lumens, tracks or the like, any of which may facilitate guidance of a tissue modification device 1032 along and/or through barrier device 1030. In various embodiments, guide feature 1035 may comprise a separate device, not attached to barrier member 1030, as in the guidewire of FIGS. 17A and 17B. Alternatively, one or more guide features 1035 may be attached to, or integral with, barrier member 1030.

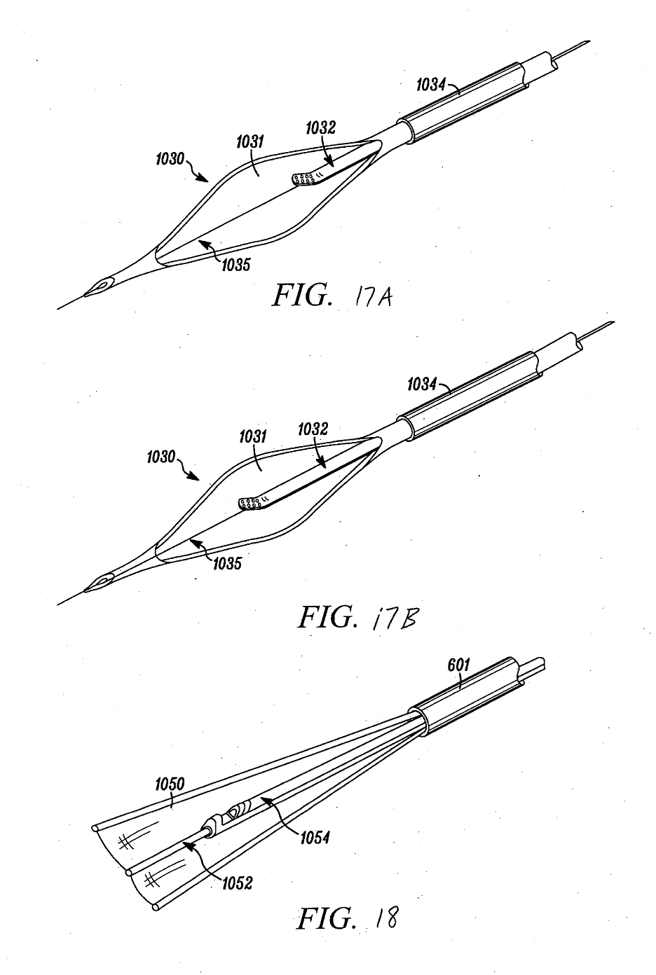

FIG. 18 shows an embodiment of a barrier device 1050 including a central rail 1052 guide member along which a tissue modification device 1054 may be guided.

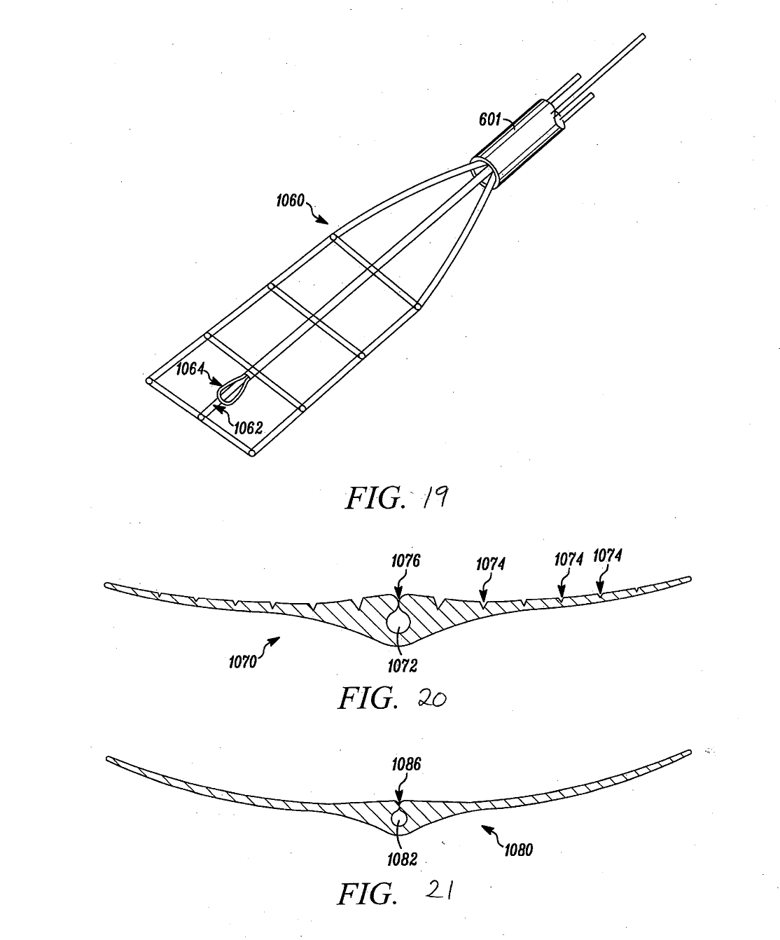

FIG. 19 shows an alternative embodiment of a barrier device 1060 including a central rail 1062 guide member along which a wire loop RF tissue modification device 1064 may be guided. In some embodiments, barrier devices 1050, 1060 and tissue modification devices 1054, 1064 may be advanced through a delivery device 601, while other embodiments may not employ such a delivery device 601.

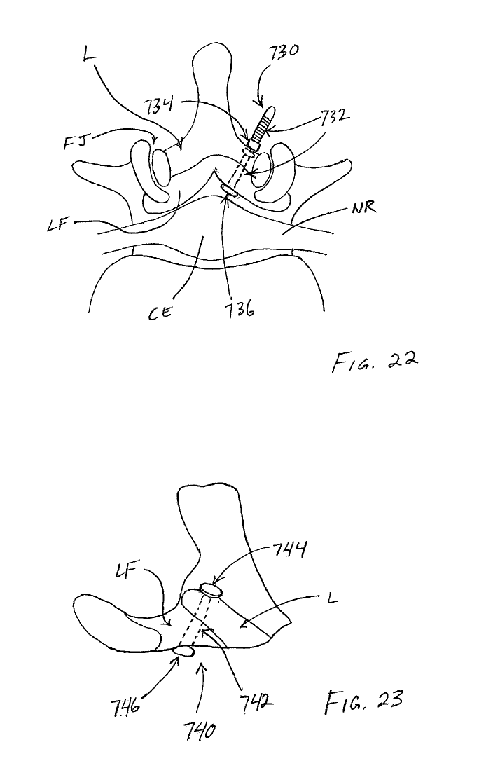

Referring to FIG. 20, in one embodiment, a barrier device 1070 may include a central channel 1072, accessible by a slit 1076, and multiple flex grooves 1074. Multiple flex grooves 1074 may facilitate collapsing of barrier device 1070.

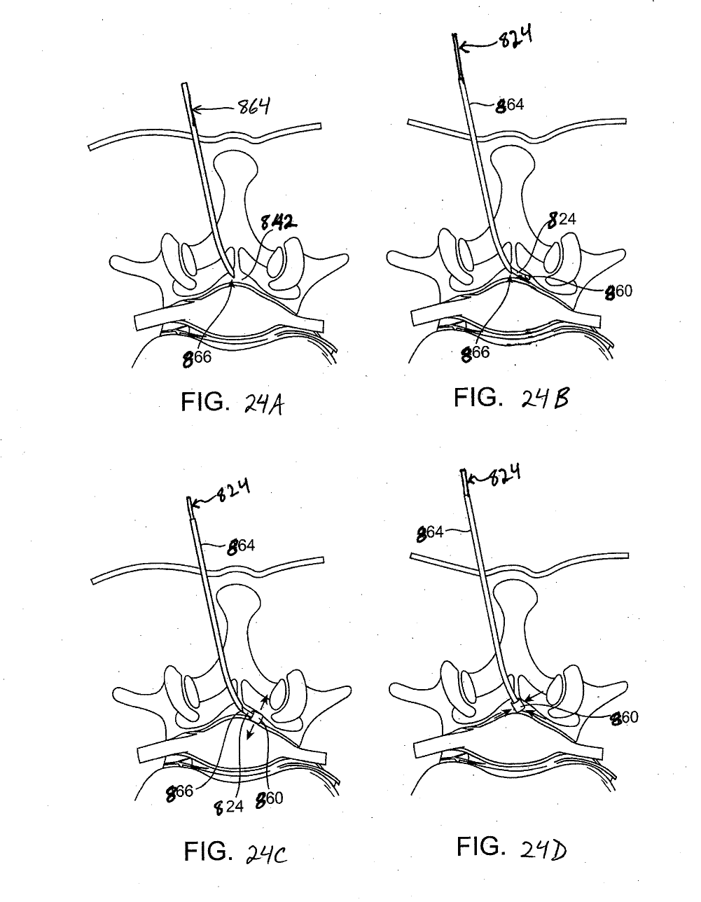

In another embodiment, as in FIG. 21, a barrier device 1080 may have a smooth, non-grooved surface and a central channel 1082, accessible by a slit 1086. Slit 1076, 1086 may facilitate coupling and decoupling of a tissue modification device with barrier device 1070, 1080. Again, for further detailed description of various barrier/shield devices, reference may be made to U.S. patent application Ser. No. 11/405,859, which was previously incorporated by reference.

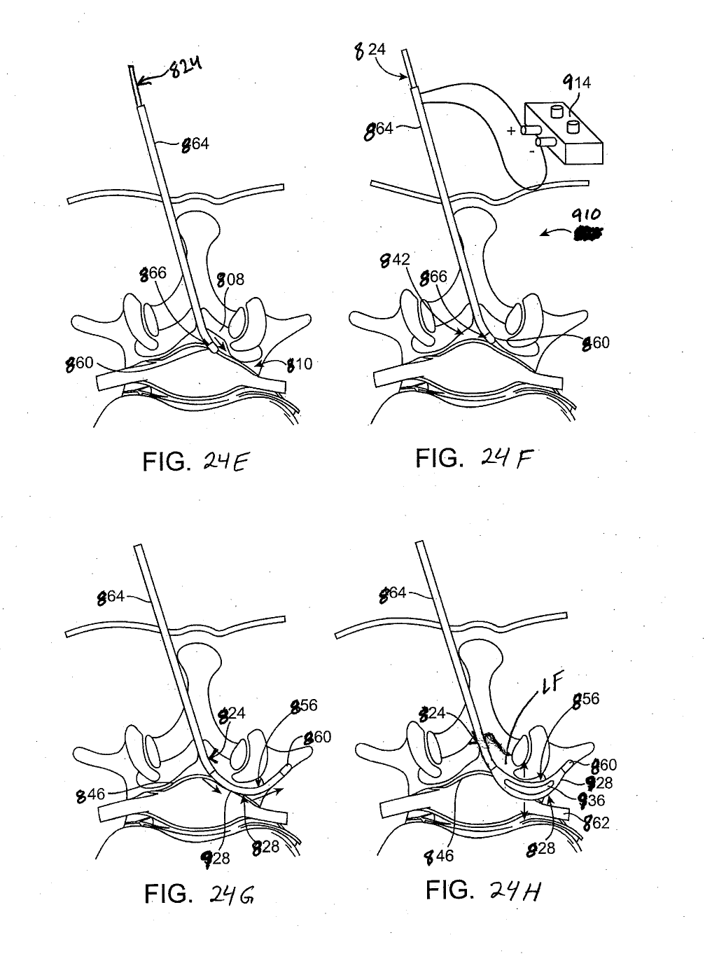

Referring now to FIG. 22, in another embodiment, a ligamentum flavum retracting device 730 may be used to help retract ligamentum flavum tissue (LF) away from cauda equina (CE) and/or nerve root (NR) tissue to alleviate spinal stenosis and/or neural/neurovascular impingement in the central spinal canal and/or lateral recess. Such a device 730 is described, for example, in U.S. patent application Ser. No. 11/251,199, which was previously incorporated by reference. Device 730 may serve to retract spinal tissue posteriorly and prevent the posterior elements, particularly the ligamentum flavum (LF), from buckling anteriorly into the spinal canal or lateral recess. Device 730 may include an anterior anchor 736, which may be placed anterior to or within the ligamentum flavum (LF), a posterior anchor 734, which may be placed posteriorly in tissue, such as posterior to a lamina (L) of a vertebra, and a body 732 extending between anchors 734, 736 to provide tension between anchors 734, 736 and thus retract ligamentum flavum (LF). In one embodiment, body 732 may include a ratcheting mechanism, such that as it is pulled back through posterior anchor 734 it increases tension between anchors 734, 736 and locks tighter and tighter.

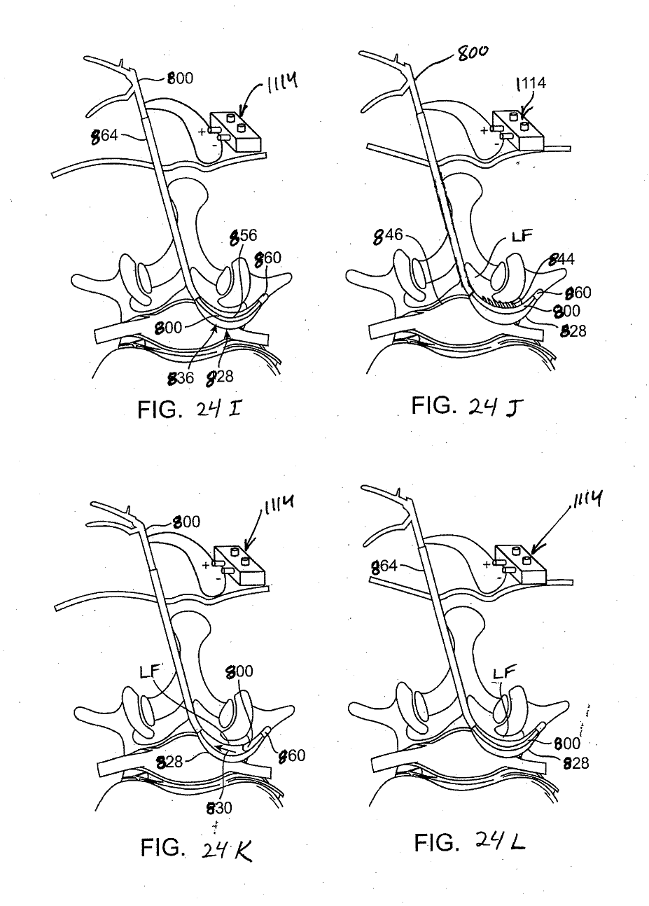

FIG. 23 illustrates a rivet-like tissue retractor device 740, which may be placed percutaneously through a hole drilled through a vertebral lamina (L). Device 740 may include an anterior anchor 746 for placement in or anterior to the ligamentum flavum (LF), a posterior anchor 744 for placement posterior to the lamina (L), and a body 742 between the two. Either of the two devices 730, 740 just described may be positioned and deployed using any suitable percutaneous technique. For example, spinal endoscopy may be used to place either ligamentum flavum retraction device 730, 740 and/or to confirm correct placement and efficacy of device 730, 740.

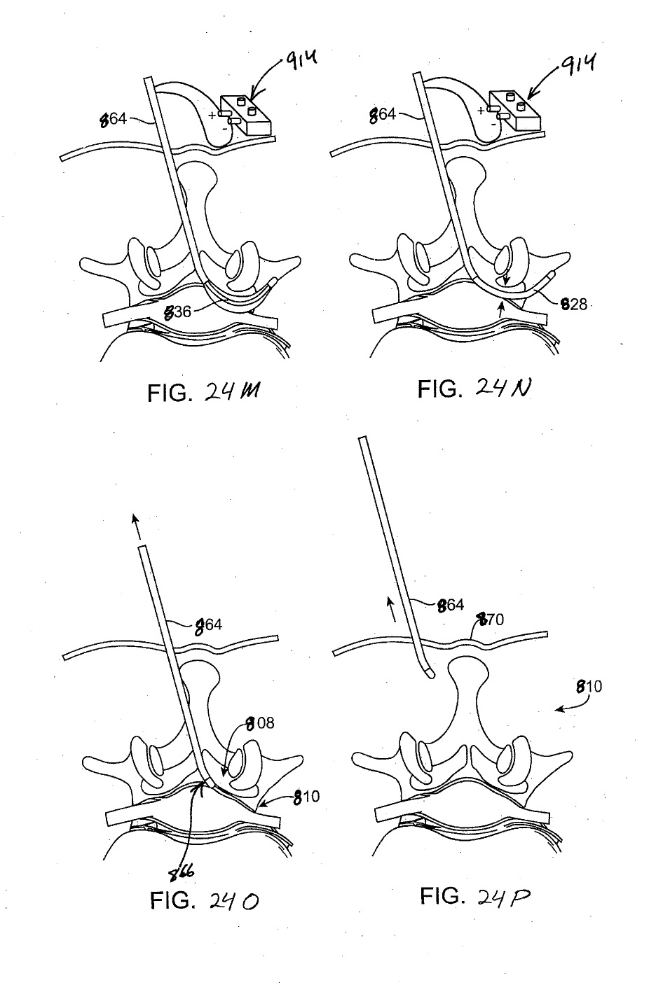

FIGS. 24A-24P demonstrate another embodiment of a method for percutaneously accessing and modifying tissue in a spine to ameliorate neural and/or neurovascular impingement and/or spinal stenosis. FIG. 24A illustrates that a percutaneous access element, such as an epidural needle 864, may be advanced percutaneously into a patient to position a sharp distal tip 866 in the epidural space 842 of the spine. For example, needle 864 may be inserted at, or one level below, the spinal interspace where tissue removal is desired. Needle 864 may be inserted into the epidural space 842 midline, ipsilateral, or contralateral to the area where the spinal canal, lateral recess and/or neuroforaminal stenosis or impingement is to be treated. In some embodiments, percutaneous access may be aided by external or internal visualization techniques, such as fluoroscopy, epidural endoscopy, combinations thereof, or the like.

In various embodiments, needle 864 may have multiple barrels or lumens. In one embodiment, for example, a first lumen may extend farther than a second lumen. In one embodiment, a first lumen and/or a second lumen may terminate in open or closed configurations at needle tip 866.

As shown in FIG. 24B, in some embodiments, a catheter 824 may be passed through needle 864 to position a distal portion of catheter 824 in the epidural space 842. The distal end of catheter 824 may include a protective hood 860 (or “cap”), which as shown in FIG. 24C, may be expanded or opened (solid-tipped arrows). As shown in FIG. 24D, with hood 860 opened, catheter 824 may be slidably retracted through needle 864 until hood 860 covers needle tip 866 (solid-tipped arrows). With hood 860 covering needle tip 866, catheter 824 may be fixed to needle 864, thus providing a blunted needle 864.

Referring to FIG. 24E, needle 864 may be advanced (solid-tipped arrow) until needle tip 866 is in a lateral recess 808, adjacent to a neural foramen 810. Needle tip 866 may be positioned adjacent the lateral recess 808, for example, by using tactile feedback from needle 864, image guidance (e.g. fluoroscopy), or combinations thereof.

In some embodiments, as shown in FIG. 24F, a neural stimulation/localization device 914 may be coupled with catheter 824, needle 864 and/or a device within catheter 824 or needle 864, such as a tissue protection barrier (not shown). Neural stimulation device 914 may comprise any currently known or hereafter invented nerve stimulation devices, may include one or more controls, and may be configured to selectively deliver and/or sense electrical current. Nerve stimulation may be used to assess and/or confirm desired placement of catheter 824 and/or needle 864 relative to nerve and target tissue. In some embodiments, catheter 824 or needle 864 may further include one or more visualization devices, such as fiber optics or other devices listed above. In some embodiments, the visualization device may be covered by a clear distal tip and may be deployed in the epidural space 842 integral with, or separate from but within, catheter 824 or needle 464.

Referring now to FIG. 24G, in one embodiment, a tissue protection barrier 828 may be passed through or with needle 864 and/or catheter 824 (solid-tipped arrows). Tissue protection barrier 828 may comprise, for example, any of the barrier devices described above or in U.S. patent application Ser. No. 11/405,859, which was previously incorporated by reference. Tissue protection barrier may be deployed into the lateral recess 808 and/or the neural foramen 810, between target tissue, such as ligamentum flavum (LF) and non-target tissue, such as dura mater 846 and associated neural (e.g., spinal cord, nerve roots, dorsal root ganglion) and neurovascular structures. In some embodiments, tissue protection barrier 828 may expand upon deployment from needle 864 to assume an atraumatic, expanded profile with rounded edges. In various embodiments, tissue protection barrier 828 may comprise a catheter, curved or straight needle, curved or straight shield, sheath, backstop, stent, net, screen, mesh or weave, panel, fan, coil, plate, balloon, accordioning panels, or combinations thereof. In some embodiments, tissue protection barrier 828 may have a tapered configuration.

In some embodiments, tissue protection barrier 828 may include a front side 856 (i.e., working side) and a back side 928 (i.e., neural protection side). Front side 856 may be electrically isolated from back side 928. Either or both of front side 856 and back side 928 may have an electrically conductive surface, and neural stimulation device 914 may be in electrical communication with either or both. In various embodiments, neural stimulation may be monitored via spinal somatosensory-evoked potentials (SSEPs), motor-evoked potentials (MEPs), and/or by looking for visual signs of muscular contraction within the extremities. SSEP, SEP, MEP or electromyogram (EMG) feedback may be monitored and/or recorded visually, and/or may be monitored audibly, potentially conveying quantitative feedback related to the volume or frequency of the auditory signal (e.g. a quantitative auditory feedback). Intensity of signal or stimulation may be monitored and used to localize the nerve during placement. Further explanation and details of various embodiments of nerve stimulation and localization methods and devices for use in spinal access are provided in U.S. patent application Ser. No. 11/429,377 (Attorney Docket No. 026445-000724US), titled “Spinal Access and Neural Localization,” and filed Jul. 13, 2006, the full disclosure of which is hereby incorporated by reference.

FIG. 24H shows tissue protection barrier 828 in its expanded configuration (solid-tipped arrows). In one embodiment, a balloon (not shown) may be inflated within tissue protection barrier 828 to cause it to expand. In some embodiments, tissue protection barrier 828 may be twisted with respect to itself, such as for positioning. In alternative embodiments, an electrical current and/or heat may be applied to the tissue protection barrier 828, which may be made from a shape memory alloy and may thus expand upon heating. In another embodiment, a spring may be positioned inside tissue protection barrier 828 to provide expansion. In yet another embodiment, tissue protection barrier 828 may comprise a spring, such as a self-expandable stent or mesh. The spring may be releasably fixed in a compressed state when the tissue protection barrier 828 is in the contracted configuration. When released, the spring may expand tissue protection barrier 828. In some embodiments, the spring may be released by a trigger mechanism. In some embodiments, expansion of tissue protection barrier 828 may apply a non-damaging pressure to the nerve branches 862. Tissue protection barrier 828 may include a window 836, which may be open in the contracted and/or expanded configuration of tissue protection barrier 828.

Referring now to FIG. 24I, a tissue removal device 800 may be slidably deployed along, through, around or over needle 864 and/or catheter 824. Tissue removal device 800 may be deployed between impinging target tissue, such as ligamentum flavum, and tissue protection barrier 828. Tissue removal device 800 may have a control handle extending from the proximal end of the needle 864. Tissue removal device 800 may be exposed to the impinging tissue through the window 836.

Tissue removal device 800 may include an energy delivery system 1114 configured to deliver RF or other energy to target tissue. Such energy may be used to ablate, vaporize, break up, combinations thereof, or otherwise change the modulus of the tissue. In various alternative embodiments, tissue removal device 800 may be configured to deliver electrical, ultrasound, thermal, microwave, laser, cryo (i.e., removing thermal energy), or combinations thereof. In one embodiment, for example, tissue removal device 800 may include one or more electrosurgery elements. The electrosurgery elements may be configured to remove and/or ablate tissue, achieve hemostasis, and/or provide neural localization in tissue adjacent to the electrosurgery elements. The electrosurgery elements may be either monopolar or bipolar RF in some embodiments. In various embodiments, the RF elements may be activated with a thermal or substantially non-thermal waveform. In other embodiments, tissue removal device 800 may include one or more lasers, high-pressure fluid devices, thermal elements, radioactive elements, textile electric conductors, conductive wire loops and/or needles configured to be used in tissue contact (e.g., needle ablation), springs, open and/or spring wire weaves, conductive polymers that can have conductive metals chemically deposited thereon, or combinations thereof.

In FIG. 24J, tissue removal device 800 is shown with multiple energy transmitting needles 844 deployed into target ligamentum flavum tissue (LF) for delivering energy. Delivered energy may alter the compression, denaturation, electrosurgical exposure, thermal remodeling (hot or cold), chemical alteration, epoxy or glues or hydrogels, and/or modulus of elasticity of the impinging tissue. For example, the modulus of elasticity of soft impinging tissue may be increased, which may improve purchase on the soft impinging tissue with the tissue removal device 800. Remodeling of the tissue during modulus alteration may alleviate impingement and obviate or reduce a need for tissue removal. Tissue removal device 800 may be designed to automatically stimulate the site of tissue removal, or have the neural stimulation and localization device 1114 stimulate the site of tissue removal, before or during tissue removal. Tissue removal device 800 may be configured to automatically stop tissue removal when nerve stimulation is sensed by the front side 856, and/or no nerve stimulation is sensed by the back side 928.

FIG. 24K illustrates that tissue removal device 800 may have one or more non-powered mechanical tissue removal elements. The non-powered mechanical tissue removal elements can be abrasives such as abrasive belts or ribbons, cutting elements such as blades, knives, scissors or saws, rongeurs, grinders, files, debriders, scrapers, graters, forks, picks, burrs, rasps, shavers, or combinations thereof.

An external activating force, for example as shown by arrow 830 (activating tissue removal) on a handle, can activate tissue removal device 800. The mechanical tissue removal elements may be used in combination or not in combination with the energy delivery device. The mechanical tissue removal elements may be pushed into and/or drawn across the impinging tissue to remove the tissue by cutting, shaving, slicing, scissoring, guillotining, scraping, tearing, abrading, debriding, poking, mutilating, or combinations thereof. The mechanical tissue removal elements (e.g., blades) may be drawn across the impinging tissue in a single direction and/or can be reciprocated. The mechanical tissue removal elements may be manually controlled and/or electronically, pneumatically or hydraulically powered. The mechanical tissue removal elements may be embedded with abrasives and/or have abrasive coatings, such as a diamond or oxide coating. Further details of various mechanical tissue modification devices are set forth above and in the patent applications incorporated by reference herein.

FIG. 24L shows tissue removal device 800 after the blade has been passed proximally to cut tissue. The blade may be passed as many times as desired, and then tissue removal device 800 may be removed through needle 864, as shown in FIG. 24M.

FIG. 24N illustrates that the tissue protection barrier 828 may be transformed into a contracted configuration (solid-tipped arrows). FIG. 240 illustrates that needle tip 866 may be translatably retracted, as shown by arrow, from the neural foramen 810 and lateral recess 808. FIG. 24P illustrates that needle 864 may be translatably withdrawn from the spine 810 and the skin 870.

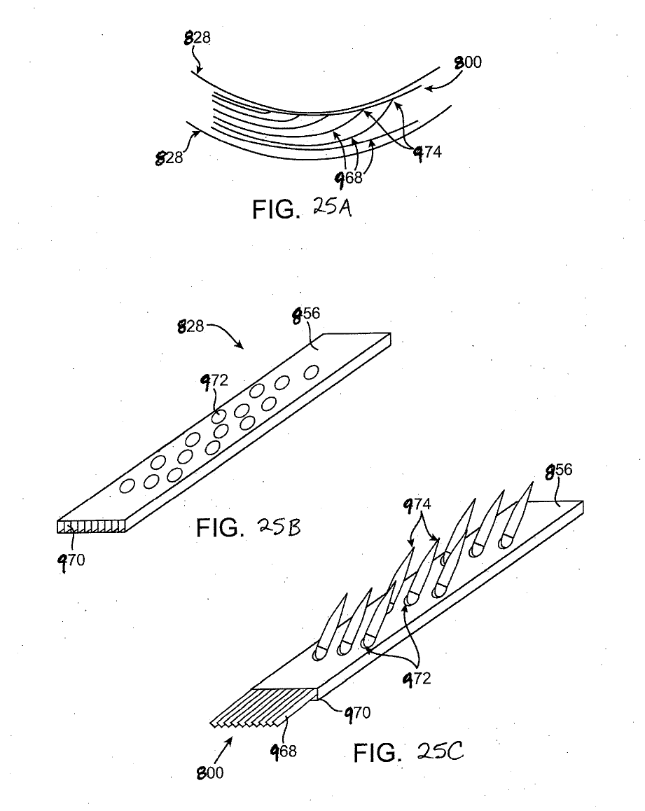

Referring now to FIGS. 25A-25C, one embodiment of a portion of a barrier 828 and tissue modifying device 800 is shown. Tissue removal device 800 may include one or more needlettes 968 and may be slidably disposed within barrier 828. Needlettes 968 may each have a needlette tip 974 and may be configured to slide out of needlette ports 972 on top surface 856 of barrier 828. In some embodiments, needlette tips 974 may be covered, coated or otherwise have a surface and/or by completely made from an electrically conductive material, and needlettes 468 may be covered, coated or otherwise have a surface made from an electrically resistive or insulating material. Needlette tips 474 may be configured to deliver electrical, ultrasound, thermal, microwave, laser and/or cryogenic energy.

In one embodiment, tissue protection barrier 528 may include multiple needlette conduits 970. Needlettes 968 may be slidably attached to needlette conduits 970. In alternative embodiments, needlettes 468 may be either solid or hollow, and in the latter case needlettes 968 may optionally be used to deliver one or more drugs or other substances to target tissue.

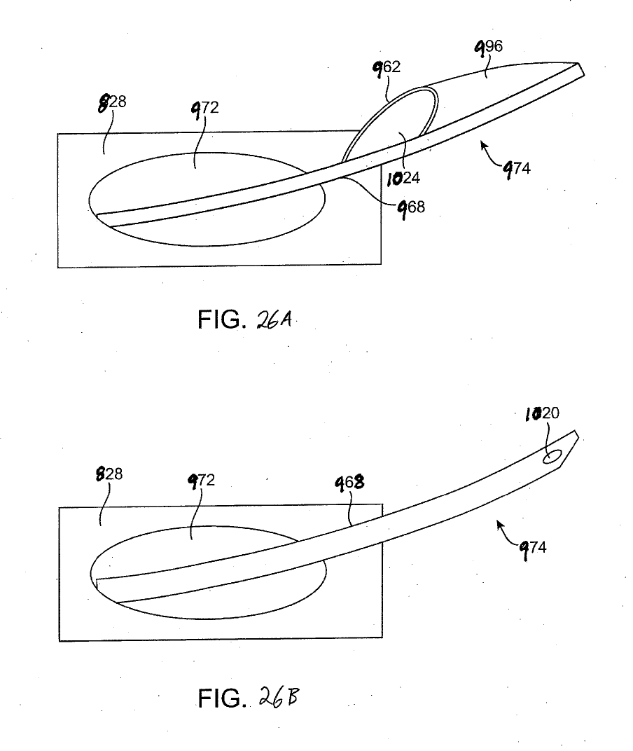

Referring now to FIG. 26A, in one embodiment, needlette tip 974 may comprise a scooped shape 996, such as a grater or shredder. Scoop 996 may have a tissue entry port 1024. Scoop 996 may be open and in fluid communication with a hollow needlette 968. Scoop 996 may have a leading edge 962, for example partially or completely around the perimeter of the tissue entry port 1024. Leading edge 962 may be sharpened and/or dulled. Leading edge 962 may be beveled. Leading edge 962 may be electrically conductive. Leading edge 962 may be configured to emit RF energy. Leading edge 962 may be a wire. Needlette tip 974 other than leading edge 962 may be electrically resistive.

In an alternative embodiment, shown in FIG. 26B, needlette tip 974 may include a tip hole 1020. Tip hole 1020 may have a sharpened perimeter. Tip hole 1020 may act as a tissue entry port. Tip hole 1050 may be in fluid communication with hollow needlette 968. Further details of these and other embodiments of tissue removal devices having needlettes and barriers having needlette ports may be found in U.S. patent application Ser. No. 11/251,199, which was previously incorporated by reference.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A method for percutaneously removing ligamentum flavum tissue in a spine to treat spinal stenosis, the method comprising:
percutaneously advancing a distal portion of a tissue removal cannula into the ligamentum flavum tissue;
uncovering a side-opening aperture disposed on the distal portion of the cannula to expose a tissue cutter disposed in the cannula; and
cutting ligamentum flavum tissue using the tissue cutter while the aperture is uncovered.

2. A method as in claim 1, wherein uncovering the aperture comprises retracting an inner cannula through the tissue removal cannula.

3. A method as in claim 1, wherein cutting ligamentum flavum tissue comprises cutting tissue using a tissue cutter selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices.

4. A method as in claim 3, wherein the ligamentum flavum tissue is cut using a radiofrequency device, and wherein the method further comprises, before the uncovering step, activating the radiofrequency device.

5. A method as in claim 1, further comprising, before the uncovering step:
articulating the distal portion of the cannula relative to the proximal portion; and
advancing the articulated distal portion at least partway into an intervertebral foramen of the spine.

6. A method as in claim 1, further comprising extending the cutter out of the aperture before the cutting step.

7. A method as in claim 1, further comprising removing the cut ligamentum flavum tissue through the cannula.

8. A method as in claim 7, wherein removing the cut tissue comprises applying suction to the cannula.

9. A method as in claim 7, wherein removing the cut tissue comprises:
engaging the cut tissue with the tissue cutter or a separate tissue engaging member; and
retracting the tissue cutter or tissue engaging member through the cannula.

10. A method as in claim 1, further comprising introducing a substance through the side-facing aperture of the cannula, the substance selected from the group consisting of a hemostatic agent, an analgesic, an anesthetic and a steroid.

11. A method as in claim 1, further comprising, before the cutting step:
activating a nerve stimulator coupled with the distal portion of the cannula; and
monitoring for response to the activation.

12. A method as in claim 1, further comprising deploying a shield between the cannula and non-target tissue before the cutting step.

13. A method as in claim 12, further comprising, before the cutting step:
activating a nerve stimulator coupled with the shield; and
monitoring for response to the activation.

14. A method for percutaneously removing ligamentum flavum tissue in a spine to treat spinal stenosis, the method comprising:
percutaneously advancing a distal portion of a tissue removal cannula into the ligamentum flavum tissue;
activating at least a first nerve stimulator coupled with the distal portion of the cannula;
monitoring for response to the activation;
uncovering a side-opening aperture disposed on the distal portion of the cannula to expose a tissue engaging member disposed in the cannula;
engaging ligamentum flavum tissue with the tissue engaging member; and
cutting ligamentum flavum tissue with a tissue cutter disposed in or on the cannula.

15. A method as in claim 14, further comprising, before the uncovering step:
activating at least a second nerve stimulator coupled with the distal portion of the cannula apart from the first nerve stimulator;
monitoring for response to activation; and
comparing an amount of activation required to illicit a response using the first nerve stimulator with an amount of activation required to illicit a response using the second nerve stimulator.

16. A method as in claim 14, wherein cutting the ligamentum flavum tissue comprises advancing an inner cannula having a sharp distal end and disposed around the tissue engaging member and within the tissue removal cannula.

17. A method for percutaneously removing ligamentum flavum tissue in a spine to treat spinal stenosis, the method comprising:
coupling a flexible distal portion of a tissue removal cannula with one end of a guidewire;
pulling the flexible distal portion into the ligamentum flavum tissue by pulling the guidewire;
uncovering a side-opening aperture disposed on the distal portion of the cannula to expose a tissue cutter disposed in the cannula; and
cutting ligamentum flavum tissue using the tissue cutter.

18. A method as in claim 17, further comprising applying tensioning force to the tissue removal cannula and the guidewire, before the cutting step, to urge the aperture against the ligamentum flavum tissue.

19. A method for percutaneously removing ligamentum flavum tissue in a spine to treat spinal stenosis, the method comprising:
percutaneously advancing a flexible distal portion of a tissue removal device into at least one of an epidural space or a ligamentum flavum of the spine;
activating an energy delivery member disposed on or in the distal portion of the tissue removal device; and
cutting ligamentum flavum tissue with the activated energy delivery member.

20. A method as in claim 19, wherein advancing the distal portion comprises pulling the distal portion behind a guidewire.

21. A method as in claim 19, wherein the distal portion is advanced at least partway into an intervertebral foramen of the spine.

22. A method as in claim 19, wherein activating the energy delivery member comprises activating a member selected from the group consisting of electrosurgical devices, bipolar electrodes, monopolar electrodes, thermal electrodes, cold ablation devices, lasers, ultrasound devices and cryogenic devices.

23. A method as in claim 19, wherein cutting the tissue comprises retracting the energy delivery member through tissue.

24. A method as in claim 19, wherein cutting the tissue comprises advancing the energy delivery member through tissue.

25. A device for percutaneously removing ligamentum flavum tissue in a spine to treat spinal stenosis, the device comprising:
a cannula having a proximal end, a tissue-penetrating distal end, and a side-facing aperture closer to the distal end than the proximal end;
an aperture cover slidably coupled with the cannula and configured to advance and retract to cover and uncover the aperture; and
a tissue cutter slidably disposed within the cannula and configured to cut ligamentum flavum tissue through the aperture while the aperture is uncovered.

26. A device as in claim 25, wherein the aperture cover comprises an inner cannula slidably disposed in the tissue removal cannula.

27. A device as in claim 25, wherein a distal portion of the cannula is articulatable relative to a proximal portion of the cannula.

28. A device as in claim 25, wherein the tissue cutter is selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices.

29. A device as in claim 25, wherein the tissue cutter is configured to extend out of the aperture.

30. A device as in claim 25, wherein the tissue cutter is configured to engage cut ligamentum flavum tissue and to be retracted through the cannula to remove the engaged tissue.

31. A device as in claim 25, further comprising a suction connector for coupling the proximal end of the cannula with a suction device for removing cut tissue through the cannula.

32. A device as in claim 25, further comprising at least a first nerve stimulator coupled with the cannula at or near the aperture.

33. A device as in claim 32, further comprising at least a second nerve stimulator coupled with the cannula, wherein the first nerve stimulator is disposed generally on the same side of the cannula as the aperture and the second nerve stimulator is disposed between about 90 degrees and about 180 degrees away from the first stimulator along a circumference of the cannula.

34. A device as in claim 25, further comprising a shield coupled with the cannula for preventing the cutter from contacting non-target tissue.

35. A device for percutaneously removing ligamentum flavum tissue in a spine to treat spinal stenosis, the device comprising:
a cannula having a proximal end, a tissue-penetrating distal end, and a side-facing aperture closer to the distal end than the proximal end;
a tissue-engaging member disposed within the cannula and adapted to engage tissue via the aperture;
an aperture cover slidably coupled with the cannula and configured to advance and retract to cover and uncover the aperture, the cover having a sharp, tissue cutting edge to cut tissue engaged by the tissue-engaging member; and
a nerve stimulation member coupled with the cannula adjacent or near the aperture.

36. A device for percutaneously removing ligamentum flavum tissue in a spine to treat spinal stenosis, the device comprising:
an elongate body having a proximal portion, a flexible distal portion, and a side-facing aperture disposed on the distal portion, wherein the distal portion is configured to be passed percutaneously into at least one of an epidural space or a ligamentum flavum of the spine; and
an energy delivery member disposed within the elongate body and configured to extend through the aperture to cut ligamentum flavum tissue.

37. A device as in claim 36, wherein the distal portion of the body is configured to pass at least partway into an intervertebral foramen of the spine.

38. A device as in claim 36, further including a guidewire coupling member disposed on the distal portion of the elongate body for pulling the distal portion into the spine.

39. A device as in claim 36, wherein the energy delivery member is selected from the group consisting of electrosurgical devices, bipolar electrodes, monopolar electrodes, thermal electrodes, cold ablation devices, lasers, ultrasound devices and cryogenic devices.

40. A device as in claim 36, wherein the energy delivery member is slidably disposed within the elongate body and is configured to be advanced through the aperture.

41. A device as in claim 40, wherein the energy delivery member comprises a wire loop electrode.

42. A device as in claim 36, wherein the elongate body further comprises a lumen through which cut ligamentum flavum tissue may be removed.

43. A device as in claim 42, further comprising a suction device couplable with the elongate body for removing the cut ligamentum flavum tissue through the lumen.

44. A device as in claim 43, further comprising an irrigation device couplable with the elongate body for passing fluid through the lumen.

45. A device as in claim 42, further comprising a substance disposed in the lumen for delivery through the aperture, wherein the substance is selected from the group consisting of a hemostatic agent, an analgesic, an anesthetic and a steroid.

46. A device as in claim 36, further comprising at least a first nerve stimulator coupled with the distal portion of the elongate body.

47. A device as in claim 46, further comprising at least a second nerve stimulator coupled with the distal portion of the elongate body apart from the first nerve stimulator.

48. A device as in claim 36, further comprising a shield coupled with the elongate body for preventing the energy delivery member from contacting non-target tissue.

49. A system for percutaneously removing ligamentum flavum tissue in a spine to treat spinal stenosis, the system comprising:
a tissue removal device, comprising:
an elongate body having a proximal portion, a flexible distal portion, and a side-facing aperture disposed on the distal portion, wherein the distal portion is configured to be passed percutaneously into at least one of an epidural space or a ligamentum flavum of the spine; and
an energy delivery member disposed within the elongate body and configured to extend through the aperture to cut ligamentum flavum tissue; and
an energy source removably couplable with the tissue removal device for supplying energy to the energy delivery member.

50. A system as in claim 49, wherein the tissue removal device further includes a guidewire coupling member disposed on the distal portion of the elongate body for pulling the distal portion into the spine.

51. A system as in claim 49, further including a guidewire configured to couple with the guidewire coupling member.

52. A system as in claim 51, further including a handle removably couplable with the guidewire for pulling the guidewire from outside a patient.

53. A system as in claim 49, wherein the elongate body further comprises a lumen through which cut ligamentum flavum tissue may be removed.

54. A system as in claim 53, further comprising a suction device for removing the cut ligamentum flavum tissue through the lumen.

55. A system as in claim 54, further comprising an irrigation device for passing fluid through the lumen.
